(19) United States
(12) Patent Application Publication
(10) Pub. No.: US 2017/0355755 A1
(43) Pub. Date: Dec. 14, 2017

(54) COMPOSITIONS AND METHODS FOR MODULATING SALM5 AND HVEM

(71) Applicant: YALE UNIVERSITY, New Haven, CT (US)

(72) Inventor: Lieping CHEN, New Haven, CT (US)

(21) Appl. No.: 15/527,384

(22) PCT Filed: Nov. 20, 2015
(86) PCT No.: PCT/US15/61752

§ 371 Date: May 17, 2017

Related U.S. Application Data
(60) Provisional application No. 62/082,730, filed on Nov. 21, 2014.

(51) Int. Cl. C07K 16/18; C07K 16/28; C07K 14/47; A61K 38/00; A61K 39/00

(52) U.S. Cl. CPC C07K 16/18; C07K 16/2875; C07K 14/47; C07K 2317/76; A61K 2039/505; A61K 38/00

(57) ABSTRACT

The invention provides compositions and methods for regulating immune responses. In one embodiment, compositions of the invention include one or more SALM5-HVEM antagonists that are able to induce inflammation in the CNS or an immune privileged tissue. In another embodiment, compositions of the invention include one or more SALM5-HVEM agonists that are able to inhibit inflammation in the CNS or an immune privileged tissue. Therapeutic uses for SALM5-HVEM antagonists and agonist are also included in the invention.

### STATEMENT REGARDING FEDERALLY SPONSORED RESEARCH OR DEVELOPMENT

This invention was made with government support under CA085721 and CA097085 awarded by the National Institutes of Health (NIH). The government has certain rights in the invention.

### BACKGROUND OF THE INVENTION

The CNS is crucial for survival and, therefore, is largely protected from the attack of inflammation and immune responses. This immune privileged status of the CNS was considered exclusively as the function of the blood-brain barrier (BBB), a unique structure largely consisting of endothelial cells and astrocytic endfeet around all capillaries within the CNS. The BBB is shown to prevent access of blood borne cells and large biological molecules such as antibodies while allow the diffusion of small metabolic products including glucose and hormones into the CNS. The permeability of BBB increases during inflammatory diseases in the CNS. More recently, however, this traditional view has been challenged by experimental findings that peripheral immune cells can cross the intact BBB (Carson et al., 2006, Immunol. Rev. 213: 48-65). Furthermore, residential cells within the CNS including neuronal and microglial cells are immune competent and could actively regulate ongoing inflammatory and immune responses in the CNS (Sternberg, 2006, Nat. Rev. Immunol. 6: 318-328; Tian et al., 2009, Trends Immunol. 30: 91-99; Tracey, 2002, Nature 420: 853-859). This regulatory mechanism of neurons could be indirect: based on neuronal modulation of T cell function through communication with residual antigen-presenting cells (APCs), which are mainly glial cells. Neurons can also execute their immune-regulatory role through direct contact with T cells (Liu et al., 2006, Nat. Med. 12: 518-525; Siffrin et al., 2010, Immunity 33: 424-436). A growing body of evidence reveals that certain infiltrating immune cells can be neuroprotective even during neuroinflammation (Hauben et al., 2000, Lancet 355: 286-287; Moalem et al., 1999, Nat. Med. 5: 49-55; Ziv et al., 2006, Nat. Neurosci. 9: 268-275). Therefore, the CNS appears to have a unique microenvironment to contain ongoing immune responses, and to maintain tissue integrity.

SALMs, also known as Lrfns (leucine-rich and fibronectin III domain-containing), are a family of newly characterized adhesion molecules. The five-member SALM family are type I transmembrane proteins predominantly expressed in the CNS and contain a typical extracellular structure composed of leucine-rich repeats (LRR), an immunoglobulin (IG) -like domain, and a fibronectin type III (FN) domain. Members of the SALM family are involved in neurite outgrowth and synapse formation (Ko et al., 2006, Neuron 50: 233-245; Morimura et al., 2006, Gene 380: 72-83; Wang et al., 2006, J. Neurosci. 26: 2174-2183). SALM1, SALM2 and SALM3, but not SALM4 or SALM5, contain an intracellular postsynaptic density-95 (PSD-95)/Discs large/zona occludens-1 (PDZ) binding domain, which recruits PDZ-95 to regulate neuron synapse formation (Wang et al., 2008, Mol. Cell Neurosci. 39: 83-94). SALM family proteins share high amino acid sequence homology, ranging from 51% to 59% identity among its members. Expression of SALM5 mRNA has been detected in parenchyma cells of the nervous system, including neurons, spinal cord and ganglia (Homma et al., 2009, Gene Expr. Patterns 9: 1-26; Ko et al., 2006, Neuron 50: 233-245; Morimura et al., 2006; Gene 380: 72-83). Consistently to the mRNA expression pattern, SALM5 protein is predominantly found in the brain and enriched in synaptic fractions (Mah et al., 2010, J. Neurosci. 30: 5559-5568). Although SALM family members are shown to regulate synapse formation and neurite outgrowth, their role in the regulation of immune responses in the CNS has not been recognized. Furthermore, binding partners of SALM family proteins remain largely unknown. An early report indicates that SALM members may form homomeric and heteromeric complexes in both heterologous cells and neurons (Seabold et al., 2008, J. Biol. Chem 283: 8395-8405). Recently, reticulon 3, a neuroendocrine-specific protein, was proposed to be a candidate for SALM5 interaction (Chang et al., 2010, J. Neurosci. Res. 88: 266-274). However, the limited localization of reticulon 3 to the endoplasmic reticulum (ER) disqualifies it as a possible physiological ligand for SALM5 on cell surface.

There is thus a need in the art for understanding molecular mechanisms underlying immune privilege and controlling immune responses in immune privileged tissues. The present invention addresses this unmet need in the art.

### BRIEF DESCRIPTION OF THE DRAWINGS

The following detailed description of preferred embodiments of the invention will be better understood when read in conjunction with the appended drawings. For the purpose of illustrating the invention, there are shown in the drawings embodiments which are presently preferred. It should be understood, however, that the invention is not limited to the precise arrangements and instrumentalities of the embodiments shown in the drawings.

FIGS. 1A-1D depict how a monoclonal antibody against SALM5 aggravates EAE. FIG. 1A depicts how SALM5 messenger RNA expression in different mouse tissues, as determined by RT-PCR. FIG. 1B is a graph depicting HEK293T cells transfected with mouse SALM5 full-length (open line) or control plasmid (filled line). The cells were stained with SALM5 mAb (clone 7A10). Data were collected and analyzed using FACS Calibur flow cytometry and FlowJo software. FIG. 1C shows the expression of SALM5 on normal spleen (panel A), brain (panel B), and spinal cord (panel C) tissues. Paraffin-embedded naive mouse tissues were stained by biotin-labeled SALM5 mAb, and the results are shown for the spleen (panel D), brain (panel E), and spinal cord (panel F) tissues. FIG. 1D is a graph depicting the clinical scores of EAE disease upon SALM5 mAb treatment. Three groups of wild type B6 mice (n=7 per group) were immunized with MOG (33-55) peptide to induce EAE. They also received control antibody, SALM5 mAb at the disease induction phase (day 1, 4), or SALM5 mAb at effector phase (day 10, 14, 17) respectively. Mice were monitored and scored for disease progression for 22 days. Results shown are representative of three independent experiments. *P<0.05 (unpaired Student's t-test).

FIGS. 2A-2G depict how SALM5 mAb increases leukocytes infiltration in the CNS without affecting T cell priming in lymphoid organs. FIG. 2A depicts the pathology of spinal cords sections from mice on day 16 after EAE induction. Inflammatory infiltrates in spinal cords were revealed by H&E staining. The infiltrates were further visualized by staining with mAb against CD3 for T cells (clone CD3-12) or MAG3 for macrophages (clone M3/84). FIGS. 2B-2C depict the quantification of infiltrating mononuclear cells in the CNS. Mice were infused with SALM5 mAb or control antibody during EAE. The mouse brains and spinal cords were prepared and extracted on day 16 after EAE induction. FIG. 2B is a graph depicting the total numbers of mononuclear cells in the CNS. FIG. 2C is a graph depicting the respective numbers of CD4+, CD8+ T cells, B cells, macrophages and microglia in the CNS. Cells were counted by flow cytometry. Data are representative of two independent experiments with five mice in each group. FIGS. 2D-2E depict draining lymph node (dLN) cells isolated and restimulated with MOG peptide (33-55) nine days after EAE induction. FIG. 2D is a graph depicting cell proliferation, which was measured by [³H] thymidine incorporation. Culture supernatants were collected at 60 hrs after restimulation. FIG. 2E is a graph depicting different cytokines levels measured by BD Cytometric Bead Array (CBA) mouse Th1/Th2/Th17 cytokine kit. Data shown are representative of two independent experiments. FIGS. 2F and 2G depict two million naïve Thy1.1+2D2 TCR transgenic T cells transferred into B6 mice one day before EAE induction. BrdU was administrated one day pre-analysis. FIG. 2F is a series of graphs depicting the total numbers of 2D2 T cells in spleens and CNS on day 15 after EAE induction, which were numerated by flow cytometry. FIG. 2G is a series of graphs depicting the division of Thy1.1+2D2 T cells in spleens and CNS as determined by BrdU incorporation on day 15 by flow cytometry analysis. Data are representative of two independent experiments with three mice in each group. *P<0.05, **P<0.01 (unpaired Student's t-test).

FIGS. 3A-3F depict enhanced inflammations in the CNS upon SALM5 mAb treatment. FIG. 3A depicts RT-PCR detection of the proinflammatory cytokines mRNA levels in the spinal cords of naïve mice or mice treated with SALM5 mAb or control antibody after EAE induction. G3PDH was served as loading control. FIG. 3B1 depicts immunohistochemistry staining of activated microglia by Iba1 expression in spinal cords on day 16 after MOG peptide immunization with SALM5 mAb or control antibody treatment. The folds of amplification in micrograph are shown in FIG. 3B2. FIG. 3C is a series of graphs depicting the expression of MHC class II and CD80 on microglia cells isolated from the CNS after EAE induction with SALM5 mAb or control antibody treatment. FIG. 3D is a graph depicting the levels of pro-inflammatory cytokines secreted by microglia/macrophages. The microglia/macrophages were isolated from the CNS from naïve, control antibody or anti-SALM5 mAb treated mice 16 days after immunization. Cells were cultured without further stimulation and the supernatant were harvested at 12 hrs. Different cytokines levels were measured by BD Cytometric Bead Array (CBA) mouse inflammatory cytokine kit. Data are representative of two independent experiments with three mice in each group. *P<0.05 (unpaired Student's t-test). FIG. 3E1 depicts the spinal cords of mice treated with SALM5 mAb or control antibody, which were intravenously injected with LPS. 24 hours later, mice were sacrificed for Iba1 staining in spinal cords. Data are representative of three mice in each group. The folds of amplification in micrograph are shown in FIG. 3E2. FIG. 3F is a series of graphs quantifying peritoneal macrophages isolated and cultured overnight with irradiated SALM5+ HEK293T cells or control HEK293T cells. LPS was added in the culture with indicated doses for 8 hrs. Culture medium was then harvested and tested for cytokines. *P<0.05 (unpaired Student's t-test).

FIGS. 4A-4E depict the identification of HVEM as the counter-receptor for SALM5. FIG. 4A depicts a set of about 2,300 plasmas encoding human transmembrane genes individually transfected into HEK293T cells in six 384-well plates and screened by purified recombinant SALM5-IG and anti-FCC FAT secondary antibody. Graphic view of individual well with positive hit for SALM5-IG were shown. FCC Receptor or OLCN transfected HEK293T cells are used as positive controls. OLCN molecule binds secondary antibody directly. FIG. 4B is a series of graphs depicting HEK293T cells transfected with Mouse HVEM, which were stained by mouse SALM5-IG at the presence of control (left panel) or HVEM mAb (right panel). FIG. 4C is a series of graphs depicting screening of the HVEM counter-receptors by CDs. HVEM-IG, which was utilized as the bait for screening the receptor-ligand proteome. The 3-D illustration represents the result of one 384-well plate. Each bar represents the total fluorescence intensity in the FL1 gate in each well. All the positive hits for HVEM-Ig were indicated, including CD160, SALM5, BTLA and SALM5. FIG. 4D is a graph depicting the interactions of human HVEM with four counter-receptors. HEK293T cells were transiently transfected to express human SALM5, BTLA, CD160 or SALM5 and were stained with human HVEM-Ig (open histograms) or control Ig (filled histograms). FIG. 4E is a series of graphs depicting the interaction of mouse HVEM with mouse counter-receptors. HEK293T cells were transiently transfected to express mouse SALM5, CD160 or SALM5 and were stained with mouse HVEM-Ig (open histograms) or control Ig (filled histograms).

FIGS. 5A-5D depict the binding analysis of HVEM-SALM5 interaction. FIG. 5A is a series of graphs depicting the competitive binding of SALM5 with other HVEM counter-receptors. HEK293T cells were transfected to express mouse HVEM, and subsequently incubated with mouse BTLA, CD160 or SALM5 recombinant fusion proteins respectively before stained by biotin-labeled mouse SALM5-Ig. FIGS. 5B1 and 5B2 are a series of graphs depicting the identification of the interacting domain on HVEM. Full-length HVEM (WT), HVEM without CRD1 domain (AHVEM), or HVEM mutants with point mutation as indicated were individually expressed on HEK293T cells and stained with SALM5-Ig (FIG. 5B2). The expression of WT and mutated HVEM were verified by HVEM polyclonal antibody staining (FIG. 5B1). FIGS. 5C-5D depict the identification of the binding domain on SALM5 for HVEM. Each extracellular domain of SALM5 including LRR, IG and FN were swapped with the corresponding domain on SALM3 by PCR cloning and fused to a c terminal EGFP. These chimeric mutants were transiently expressed on HEK293T cells and stained by mouse HVEM-Ig. FIG. 5C is an illustration depicting positive (+) or negative (−) binding to mouse. FIG. 5D is a series of graphs depicting a summary of the binding assay by flow cytometry.

FIGS. 6A-6D depict how SALM5 interacts with HVEM to inhibit EAE. FIG. 6A is a series of graphs depicting splenocytes from wild type (wt) or HVEM−/−mice, which were stained with biotin-conjugated mouse SALM-Ig (open) or FLAG-Ig (close). Cells were gated for lineage-specific markers as indicated. FIG. 6B is a series of graphs depicting how anti-SALM5 mAb (clone 7A10) blocks SALM5-HVEM interaction. HEK293T cells were transiently transfected with the plasmid encoding mouse SALM5 (open) or control plasmid (close). HEK293T transfectants were pre-incubated with control antibody or anti-SALM5 mAb before stained with mouse HVEM-Ig. FIG. 6C is a series of graphs depicting the mapping of the binding domain for anti-SALM5 (clone 7A10) on SALM5. Chimeras for mouse SALM5 and SALM3 described in FIG. 5C were transiently expressed on HEK293T cells and stained by anti-SALM5 mAb (clone 7A10). FIG. 6D is a series of graphs depicting the role of endogenous HVEM for anti-SALM5 mAb effect in EAE model. Wild-type (WT) and HVEM-knockout mice (HVEM−/−) were immunized with MOG (33-55) peptide to induce EAE. SALM5 mAb or control antibody was given from the beginning of the experiment (n=7). Clinical scores of EAE diseases were measured daily. Representative results from two independent experiments are shown. *P<0.05 (unpaired Student's t-test).

FIG. 7 depicts immunostaining of SALM5 in normal mouse brain sections by different mouse SALM5 antibodies. Tissues were deparaffinized and rehydrated prior to Ag retrieval in citrate buffer. Tissues were then stained with different SALM5 antibodies, followed by incubation with amplification system k1500 (DakoCytomation, Glostrup, Denmark). After HRP staining, slides were visualized with 3-3′ diaminobenzidine (Sigma Aldrich, St. Louis, Mo.).

FIG. 8 is a graph depicting how administration of F(ab)'2 of SALM5 mAb aggravates EAE disease. Two group of wild type B6 mice (n=7 per group) were immunized with MOG (33-55) peptide to induce EAE as described in the Materials and Methods. Mice also received 200 ug/mouse control antibody or F(ab)'2 of SALM5 mAb on day 9, 11, 13, respectively. Mice were monitored and scored for disease progression up to 22 days. *P<0.05 (unpaired Student's t-test).

FIGS. 9A-9E depict how administration of SALM5 mAb does not affect T cell differentiation. FIG. 9A is a series of graphs depicting CD4+ T cells isolated from CNS of mice with EAE which were intracellularly stained for IL-17, IFN-γ upon five hours stimulation of PMA. FIG. 9B is a series of graphs depicting the percentages of regulatory T cells (Foxp3-positive) in CD4+ T cells of CNS, which were directly analyzed upon mononuclear cells isolation. In FIGS. 9A-9B, mononuclear cells were isolated from brain and spinal cord of mice with EAE. FIG. 9C is a series of graphs depicting the percentages of IL-17 and IFN-γ-producing cells in CD4+ T cells measured by intracellular flow cytometry 72 hours after cultured with MOG peptide. FIG. 9D is a series of graphs depicting the percentage of Treg cells in dLN T cells directly analyzed by intracellular Foxp3 staining right after isolation. Data are representative of three independent experiments. Nine days after EAE induction, draining lymph node (dLN) cells were isolated and restimulated with MOG peptide (33-55). FIG. 9E is a graph depicting splenocytes from control or anti-SALM5 mAb treated mice 12 days after EAE induction, which were harvested and restimulated with MOG 33-55 in the presence of IL-12 and IL-2 for 3-4 days. Live cells were i.v. transferred into B6 Rag KO mice, which were later treated with PT. Mice were monitored and scored for disease progression

FIGS. 10A-10B depict how SALM5 did not directly affect T cell proliferation. FIG. 10A is a graph depicting purified TCR transgenic 2D2 T cells stimulated by coated anti-CD3 mAb in the presence of coated SALM5-Ig or control Ig. T cell proliferation, which was analyzed by [³H]-thymidine incorporation assay. FIG. 10B is a series of graphs depicting CFSE-labeled OT-1 T cells, which were cultured with irradiated Kb-OVA-HEK293T cells, which were transfected with control plasmid or mouse SALM5 pcDNA. Numbers shown represent percentages of OT-1 cells with more than one division. Data are representatives of two independent experiments.

FIGS. 11A-11B depicts CDs screening of a library of transmembrane proteins. A set of about 2,300 expression plasmids for human transmembrane genes were individually seeded into five 384-well plates, and transiently transfected into 293T cells by lipofectamine. SALM-Ig or HVEM-Ig (R&D systems, Minneapolis, Minn.) and anti-human Ig FMAT blue secondary antibody (Applied Biosystems, Foster City, Calif.) were added into the wells 8 hours after transfection. The plates were read 24 hrs after transfection by the Applied Biosystems 8200 cellular detection system and analyzed by CDS 8200 software. The 3-D illustration (FIG. 11A) represents the results of five 384-well plates. Each plate contains a well of FcR plasmid as an internal positive control for transfection and fusion protein. FIG. 11A is a 3-D illustration of five 384-well plates screened by SALM5-Ig. FIG. 11B is a series of images depicting a graphic view of individual well with positive hit for HVEM-Ig.

FIGS. 12A-12C are a series of graphs depicting binding of the mouse BTLA and CD160 binding by HVEM mutants. Wild type HVEM and HVEM mutants were transiently expressed on HEK293T cells. Cells were stained by BTLA-Ig (FIG. 12B) or CD160-Ig fusion protein (FIG. 12C) 24 hrs after transfection. The expression level of HVEM individual was indicated by polyclonal HVEM antibody staining (FIG. 12A).

FIGS. 13A-13B depict HVEM expression on various cell types and the blocking effect of anti-SALM5 mAb. FIG. 13A is a series of graphs depicting splenocytes and microglia isolated from naïve B6 (open) or HVEM−/− (close) mice, which were stained for HVEM mAb together with the mAb against different cell surface markers as indicated elsewhere herein. FIG. 13B is a series of graphs depicting isolated microglia from naïve B6 mice stained by biotin-conjugated mouse SALM-Ig, which was pre-incubated with control or anti-SALM5 mAb.

FIGS. 14A-14B depict the effect of SALM5 mAb in EAE disease upon intrathecally injection. B6 mice were immunized with MOG (33-55) peptide to induce EAE. FIG. 14A is a graph depicting the EAE score of mice treated with control or SALM5 mAb 150 ug/mouse intracranially two days after immunization. FIG. 14B is a graph depicting the EAE score of B6 Rag KO mice adaptively transferred with 1*10⁶ wt or HVEM Knockout (KO) 2D2 transgenic T cells. After two weeks, mice were induced with EAE and treated with control or SALM5 mAb respectively. Mice were monitored and scored for disease progression for 22 days.

FIG. 15 is a series of graphs depicting the effects of SALM5 and SALM5 on macrophage activation. Peritoneal macrophages were isolated and cultured overnight with irradiated control HEK293T cells, SALM5 or SALM5 transfectants. 5 ng/ml LPS was added in the culture with indicated doses for 12 hrs. Culture medium was then harvested and tested for cytokines. *P<0.05 (unpaired Student's t-test).

FIG. 16 is a graph depicting how administration of SALM5-Ig aggravates EAE diseases. Mice were immunized with MOG (33-55) peptide to induce EAE. Two days after immunization, mice were hydrodynamically injected with control or SALM5-Ig plasmids to express proteins in vivo. Mice were monitored and scored for disease progression for at least 22 days.

FIG. 17 is a series of graphs depicting how administration of anti-SALM5 does not affect graft-versus-host (GVH) response. BDF1 mice were injected intravenously with 5×10⁷ spleen cells from B6 mice. Mice were treated with control or anti-SALM5 mAb 400 ug/mouse on day 1 and day 3 after cell transferring. Seven days later, spleens from the BDF1 recipients were harvested and stained with anti-H-2K^{d} mAb, together with either anti-CD8 or anti-CD19 mAb. Percentage of donor (H-2K^{d}-negative) CD8⁺ T cells or recipient (H-2K-positive) CD19+ B cells in total spleen was shown in the panel.

### DETAILED DESCRIPTION

The present invention is partly based on the identification and characterization of a pathway which modulates inflammatory immune responses in an immune-privileged tissue. For example, SALM5 a member of the synaptic adhesion-like molecule (SALM) found mainly on neuronal cells in the CNS, was discovered to interact with Herpes virus entry mediator (HVEM) on myeloid cells to suppress inflammation in the CNS during ongoing neuroinflammation.

Accordingly, the invention provides compositions and methods for targeting a negative regulator of inflammation in an immune privileged tissue, such as the CNS, testes, placenta and eye. In one embodiment, the negative regulatory of inflammation includes SALM5, HVEM, as well as their functional equivalents. That is, the invention is based on the discovery of the direct link between the binding of SALM5 and HVEM with an inflammatory response whereby inhibiting the SALM5/HVEM binding inhibits the immunosuppressive role of SALM5. By inhibiting one or more of SALM5, HVEM, and functional equivalents thereof, inflammation can be induced. Alternatively, promoting the interaction between SALM5 and HVEM allows SALM5 to exhibit its immunosuppressive role in the immune privileged tissue and therefore suppress inflammation in order to promote an immune privileged environment.

In one embodiment, the invention provides compositions and methods for modulating the interaction between SALM5 and HVEM in an immune privileged tissue. In one embodiment, inhibition of the binding of SALM5 with HVEM in the immune privileged tissue increases inflammation in the immune privileged tissue. In another embodiment, promoting the interaction between SALM5 and HVEM in the immune privileged tissue suppresses inflammation in the immune privileged tissue. In various embodiments, the immune privileged tissue is at least of the CNS, the testes, the placenta and the eye.

In one embodiment, the invention provides compositions and methods for regulating CNS diseases. This is because the invention provides a way to regulate the immune response in the CNS.

In one embodiment, the inhibitor of the invention that is able to inhibit the interaction between SALM5 and HVEM is an antibody that specifically binds to SALM5. In one embodiment, the SALM5 antibody binds SALM5 by way of its leucine-rich repeat (LRR) domain. In another embodiment, the inhibitor of the invention that is able to inhibit the interaction between SALM5 and HVEM is an antibody that specifically binds to HVEM. In some embodiments, the HVEM antibody interacts with the CRD1 domain on HVEM. In yet another embodiment, the antibodies of the invention do not affect T cell activation outside the CNS.

In one embodiment, the invention provides a method of disrupting the interaction between SALM5 and HVEM to enhance inflammatory immune responses in order to treat a neoplasm in the CNS. In another embodiment, disrupting the interaction between SALM5 and HVEM to enhance inflammatory immune responses allows for treating a viral infection in the CNS.

### Definitions

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of±20%, ±10%,±5%,±1%, or±0.1% from the specified value, as such variations are appropriate to perform the disclosed methods.

The term “abnormal” when used in the context of organisms, tissues, cells or components thereof, refers to those organisms, tissues, cells or components thereof that differ in at least one observable or detectable characteristic (e.g., age, treatment, time of day, etc.) from those organisms, tissues, cells or components thereof that display the “normal” (expected) respective characteristic. Characteristics which are normal or expected for one cell or tissue type, might be abnormal for a different cell or tissue type.

The phrase “activator,” as used herein, means to enhance a molecule, a reaction, an interaction, a gene, an mRNA, and/or a protein's expression, stability, function or activity by a measurable amount.

By “acute inflammatory response” is meant a short-term activation of the immune system in a subject. The short-term activation of the immune response in the subject desirably is present for less than one month, for example, less than two weeks, less than one week, less than one day, less than 12 hours, or even less than 6 hours. In a desirable embodiment, the acute inflammatory response requires the activation of macrophages.

The term “antibody,” as used herein, refers to an immunoglobulin molecule which is able to specifically bind to a specific epitope on an antigen. Antibodies can be intact immunoglobulins derived from natural sources or from recombinant sources and can be immunoreactive portions of intact immunoglobulins. The antibodies useful in the present invention may exist in a variety of forms including, for example, polyclonal antibodies, monoclonal antibodies, intracellular antibodies (“intrabodies”), Fv, Fab and F(ab)₂, as well as single chain antibodies (scFv), camelid antibodies and humanized antibodies (Harlow et al., 1999, Using Antibodies: A Laboratory Manual, Cold Spring Harbor Laboratory Press, N.Y.; Harlow et al., 1989, Antibodies: A Laboratory Manual, Cold Spring Harbor, N.Y.; Houston et al., 1988, Proc. Natl. Acad. Sci. USA 85: 5879-5883; Bird et al., 1988, Science 242: 423-426).

As used herein, the term “heavy chain antibody” or “heavy chain antibodies” comprises immunoglobulin molecules derived from camelid species, either by immunization with an antigen and subsequent isolation of sera, or by the cloning and expression of nucleic acid sequences encoding such antibodies. The term “heavy chain antibody” or “heavy chain antibodies” further encompasses immunoglobulin molecules isolated from an animal with heavy chain disease, or prepared by the cloning and expression of V_{H} (variable heavy chain immunoglobulin) genes from an animal.

By the term “synthetic antibody” as used herein, is meant an antibody which is generated using recombinant DNA technology, such as, for example, an antibody expressed by a bacteriophage as described herein. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art.

The term “antigen” or “Ag” as used herein is defined as a molecule that provokes an immune response. This immune response may involve either antibody production, or the activation of specific immunologically-competent cells, or both. The skilled artisan will understand that any macromolecule, including virtually all proteins or peptides, can serve as an antigen. Furthermore, antigens can be derived from recombinant or genomic DNA. A skilled artisan will understand that any DNA, which comprises a nucleotide sequences or a partial nucleotide sequence encoding a protein that elicits an immune response therefore encodes an “antigen” as that term is used herein. Furthermore, one skilled in the art will understand that an antigen need not be encoded solely by a full length nucleotide sequence of a gene. It is readily apparent that the present invention includes, but is not limited to, the use of partial nucleotide sequences of more than one gene and that these nucleotide sequences are arranged in various combinations to elicit the desired immune response. Moreover, a skilled artisan will understand that an antigen need not be encoded by a “gene” at all. It is readily apparent that an antigen can be generated synthesized or can be derived from a biological sample. Such a biological sample can include, but is not limited to a tissue sample, a tumor sample, a cell or a biological fluid.

“An antigen presenting cell” (APC) is a cell that are capable of activating T cells, and includes, but is not limited to, monocytes/macrophages, B cells and dendritic cells (DCs).

The term “binding” refers to a direct association between at least two molecules, due to, for example, covalent, electrostatic, hydrophobic, ionic and/or hydrogen-bond interactions under physiological conditions.

By “cytokine response” is meant an increase in expression or activity of a cytokine in a subject or in cell culture. An exemplary cytokine response involves induction or activation of Tumor Necrosis Factor alpha (TNFα), IL-6, IL-8, or IL-10. Desirably, a cytokine response also involves p38 MAP kinase, erk1/2, or NF-κB activation.

By “chronic inflammatory response” is meant the prolonged activation of the immune system in a subject. The activation of the immune system desirably is at least 3 months, 6 months, 1 year, 5 years, 10 years, or even life-long. A chronic inflammatory response preferably involves the induction of cytokines.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

A disease or disorder is “alleviated” if the severity of a symptom of the disease or disorder, the frequency with which such a symptom is experienced by a patient, or both, is reduced.

An “effective amount” or “therapeutically effective amount” of a compound is that amount of compound which is sufficient to provide a beneficial effect to the subject to which the compound is administered. An “effective amount” of a delivery vehicle is that amount sufficient to effectively bind or deliver a compound.

By the term “immune reaction,” as used herein, is meant the detectable result of stimulating and/or activating an immune cell.

“Immune response,” as the term is used herein, means a process that results in the activation and/or invocation of an effector function in either the T cells, B cells, natural killer (NK) cells, and/or antigen-presenting cells (APCs). Thus, an immune response, as would be understood by the skilled artisan, includes, but is not limited to, any detectable antigen-specific or allogeneic activation of a helper T cell or cytotoxic T cell response, production of antibodies, T cell-mediated activation of allergic reactions, macrophage infiltration, and the like.

“Immune cell,” as the term is used herein, means any cell involved in the mounting of an immune response. Such cells include, but are not limited to, T cells, B cells, NK cells, antigen-presenting cells (e.g., dendritic cells and macrophages), monocytes, neutrophils, eosinophils, basophils, and the like.

By the term “an inhibitor of the interaction of SALM5 with HVEM,” as used herein, is meant any compound or molecule that detectably inhibits the interaction of SALM5 with HVEM, or otherwise interferes with signaling via SALM5 and/or HVEM.

As used herein, an “inhibitory-effective amount” is an amount that results in a detectable (e.g., measurable) amount of inhibition of an activity associated with the interaction between SALM5 with HVEM.

As used herein, an “instructional material” includes a publication, a recording, a diagram, or any other medium of expression which can be used to communicate the usefulness of a compound, composition, vector, or delivery system of the invention in the kit for effecting alleviation of the various diseases or disorders recited herein. Optionally, or alternately, the instructional material can describe one or more methods of alleviating the diseases or disorders in a cell or a tissue of a mammal. The instructional material of the kit of the invention can, for example, be affixed to a container which contains the identified compound, composition, vector, or delivery system of the invention or be shipped together with a container which contains the identified compound, composition, vector, or delivery system. Alternatively, the instructional material can be shipped separately from the container with the intention that the instructional material and the compound be used cooperatively by the recipient.

By the term “modulating” an immune response, as used herein, is meant mediating a detectable increase or decrease in the level of an immune response in a mammal compared with the level of an immune response in the mammal in the absence of a treatment or compound, and/or compared with the level of an immune response in an otherwise identical but untreated mammal. The term encompasses perturbing and/or affecting a native signal or response thereby mediating a beneficial therapeutic response in a mammal, preferably, a human.

By the term “modulating” central nervous system function, as used herein, is meant mediating a detectable increase or decrease in the function of the central nervous system in a mammal compared with the level of central nervous system function in the mammal in the absence of a treatment or compound, and/or compared with the level of central nervous function in an otherwise identical but untreated mammal. The term encompasses perturbing and/or affecting a native signal or response thereby mediating a beneficial therapeutic response in a mammal, preferably, a human. In some instances, modulating central nervous system function is associated with modulating the activity of cells of the central nervous system. An example of a cell of the central nervous system is a neuron.

As used herein, a “modulator of SALM5 and HVEM interaction” is a compound that binds to SALM5, HVEM, or both and modifies the activity or biological function of SALM5, HVEM, or both as compared to the activity or biological function of SALM5, HVEM, or both in the absence of the modulator. The modulator may be a receptor agonist, which is able to activate the receptor and cause a biological response that is enhanced over the baseline activity of the unbound receptor. The modulator may be a partial agonist, which does not activate the receptor thoroughly and causes a biological response that is smaller in magnitude compared to those of full agonists. The modulator may be a receptor antagonist, which binds to the receptor but does not activate it, resulting in receptor blockage and/or inhibition of the binding of other agonists. The modulator may be an inverse agonistic, which reduces the activity of the receptor by inhibiting its constitutive activity.

“Naturally-occurring” as applied to an object refers to the fact that the object can be found in nature. For example, a polypeptide or polynucleotide sequence that is present in an organism (including viruses) that can be isolated from a source in nature and which has not been intentionally modified by man is a naturally-occurring sequence.

The terms “patient,” “subject,” “individual,” and the like are used interchangeably herein, and refer to any animal, or cells thereof whether in vitro or in situ, amenable to the methods described herein. In certain non-limiting embodiments, the patient, subject or individual is a human.

As used herein, the term “SALM5-HVEM antagonist” refers to a compound that inhibits, reduces, or blocks the biological activity or expression of SALM5 and/or HVEM. Suitable SALM5-HVEM antagonists include, but are not limited to, antibodies and antibody fragments, polypeptides including fragments of SALM5 or HVEM, small organic compounds, and nucleic acids.

As used herein, the term “SALM5-HVEM agonist” refers to a compound that increases the biological activity or expression of SALM5 and/or HVEM. Suitable SALM5-HVEM antagonists include, but are not limited to, antibodies and antibody fragments, polypeptides, small organic compounds, and nucleic acids.

As used herein, the phrase that a molecule “specifically binds” to a target refers to a binding reaction which is determinative of the presence of the molecule in the presence of a heterogeneous population of other biologics. Thus, under designated immunoassay conditions, a specified molecule binds preferentially to a particular target and does not bind in a significant amount to other biologics present in the sample. Specific binding of an antibody to a target under such conditions requires the antibody be selected for its specificity to the target. A variety of immunoassay formats may be used to select antibodies specifically immunoreactive with a particular protein. For example, solid-phase ELISA immunoassays are routinely used to select monoclonal antibodies specifically immunoreactive with a protein.

A “therapeutic” treatment is a treatment administered to a subject who exhibits signs or symptoms of pathology, for the purpose of diminishing or eliminating those signs or symptoms.

As used herein, “treating a disease or disorder” means reducing the frequency or severity with which a sign or symptom of the disease or disorder is experienced by a patient.

The phrase “therapeutically effective amount,” as used herein, refers to an amount that is sufficient or effective to prevent or treat (delay or prevent the onset of, prevent the progression of, inhibit, decrease or reverse) a disease or disorder, including alleviating signs and symptoms of such diseases and disorders.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### Description

The invention is based on the discovery that Herpes virus entry mediator (HVEM) is the counter-receptor for synaptic adhesion-like molecule 5 (SALM5). The disclosure presented herein demonstrates that the interaction of SALM5 and HVEM contributes to the immune suppressive aspects of the CNS. The findings presented herein provide a unique mechanism reducing immune responses in immune privileged tissues and provide new therapeutic targets for the control of immune responses in immune privileged tissues.

Accordingly, the invention provides compositions and methods for targeting a negative regulator of inflammation in the CNS or an immune privileged tissue. The negative regulatory of inflammation includes but is not limited to SALM5, HVEM, as well as their functional equivalents.

The present invention provides compounds and methods for modulating an immune response (e.g., inflammation) in the CNS or other immune privileged tissue. In one embodiment, inflammation is enhanced by disrupting the interaction between SALM5 and HVEM. In another embodiment, inflammation is inhibited by promoting or mimicking the interaction between SALM5 and HVEM. Accordingly, CNS diseases can be treated using the compounds of the invention.

The invention also provides compounds and methods of modulating downstream targets of SALM5, HVEM, or both as well as their functional equivalents. Diseases mediated by SALM5, HVEM, or both as well as their functional equivalents include, but are not limited to, diseases characterized in part by abnormalities in cell proliferation (e.g., neoplasm, cancer, tumor growth), programmed cell death (apoptosis), cell migration and invasion, and angiogenesis associated with tumor growth.

### Inhibitory Compositions

As described elsewhere herein, the invention is based on the discovery that inhibition of SALM5, HVEM, or both as well as their functional equivalents and interactions thereof contributes to the regulation of inflammation in the CNS. This observation is the first of its kind by providing a direct link between the pathway involving SALM5 and the negative regulation and control of neuroinflammation. As such, the invention provides for a way to therapeutically inhibit the negative regulation and control of inflammation in the CNS or other immune privileged tissue. This inhibition of a negative regulator of inflammation can be beneficial, in addition to other effects, by inhibiting the activity or expression of SALM5, HVEM, or both as well as their functional equivalents and interactions thereof in order to promote inflammation in the CNS or other immune privileged tissue.

Based on the disclosure herein, the present invention includes a generic concept for inhibiting a negative regulator of inflammation or any component of the signal transduction pathway associated with the interaction between SALM5 and HVEM or functional equivalents thereof. Preferably, the negative regulator of inflammation includes SALM5, HVEM and functional equivalents thereof, whereby inhibiting any one or more of these proteins is associated with increasing inflammation in the CNS or other immune privileged tissue. Such an increase in inflammation is useful in the treatment of, for example, neoplasm (e.g., cancer, tumor, etc.) or infection (e.g., bacterial, viral, fungal, protozoan, etc.), in an immune privileged tissue.

In one embodiment, the invention comprises a composition for enhancing inflammation in the CNS or other immune privileged tissue. The composition comprises an inhibitor of any one or more of the following modulators of inflammation: SALM5, HVEM and functional equivalents thereof. The composition comprising the inhibitor includes but is not limited to a small interfering RNA (siRNA), a microRNA, an antisense nucleic acid, a ribozyme, an expression vector encoding a transdominant negative mutant, an intracellular antibody, an antibody, a polypeptide, a peptide, a small molecule, and the like.

Antagonists or Inhibitors of SALM5-HVEM Interaction

SALM5-HVEM antagonists that reduce or inhibit the binding of SALM5 to HVEM can inhibit the suppressive effects of SALM5 on inflammation. Accordingly, SALM5-HVEM antagonists of the invention can promote inflammation in the CNS or other immune privileged tissue. In one embodiment, SALM5-HVEM antagonists of the invention reduce the binding of SALM5-HVEM by, directly or indirectly, reducing the binding of the LRR domain of SALM5 to the CRD1 domain of HVEM. The reduction in binding between SALM5 and HVEM can be by less than 50%, 40%, 30%, 20%, 10%, 5% or less, as compared to a control. SALM5-HVEM antagonists can be competitive or non-competitive inhibitors of SALM5-HVEM binding. SALM5-HVEM antagonists that reduce or inhibit the binding of SALM5 to HVEM include antibodies and antibody fragments that bind HVEM or SALM5, SALM5 or HVEM recombinant peptides or polypeptides including soluble fragments of SALM5 or HVEM, SALM5 or HVEM decoy peptides or polypeptides including soluble fragments of SALM5 or HVEM, and small organic compounds.

In one embodiment, SALM5-HVEM antagonists that bind to SALM5 or HVEM reduce or inhibit the interaction between SALM5 and HVEM by at least 20%, more preferably by at least 30%, more preferably by at least 40%, 50%, 60%, 70%, 80%, 90%, 95%, 96%, 97%, 98%, 99% or more.

In one embodiment, SALM5-HVEM antagonists that are capable of binding to SALM5 or HVEM do not increase SALM5 or HVEM activity in a cell expressing SALM5 or HVEM on its surface. In some embodiments SALM5-HVEM antagonists are capable of reducing or inhibiting one or more activities of SALM5 or HVEM in a cell expressing SALM5 or HVEM on its surface. In some embodiments, the cell expressing HVEM is a lymphocyte, a T cell, a CD4+ T cell, a CD8+ T cell, a Th1 cell, a B cell, a plasma cell, a macrophage, or an NK cell. In preferred embodiments, the cell expressing HVEM is a myeloid cell. In some embodiments, the cell expressing SALM5 is a cell of the CNS, preferably a neuronal cell. SALM5-HVEM antagonists that bind to SALM5 or HVEM reduce or inhibit one or more SALM5 or HVEM activities by at least 20%, more preferably by at least 30%, more preferably by at least 40%, 50%, 60%, 70%, 80%, 90%, 95%, 96%, 97%, 98%, 99% or more.

Inhibitory Antibodies

In one embodiment, SALM5-HVEM antagonists are antibodies. Antibodies or antibody fragments that specifically bind to SALM5 or HVEM can be used to reduce or inhibit the binding of SALM5 to HVEM. Methods of producing antibodies are well known and within the ability of one of ordinary skill in the art and are described in more detail elsewhere herein.

The antibodies disclosed herein specifically bind to a SALM5 or an HVEM protein and are capable of reducing or inhibiting the binding of SALM5 to HVEM. These antibodies are defined as “blocking,” “function-blocking” or “antagonistic” antibodies. In some embodiments the antagonistic antibodies specifically bind to a portion of the extracellular domain of SALM5 or HVEM. In other embodiments, the antagonistic antibodies specifically bind to the LRR domain of SALM5 or the CRD1 domain of HVEM.

As will be understood by one skilled in the art, any antibody that can recognize and bind to an antigen of interest is useful in the present invention. That is, the antibody can inhibit a negative regulator of inflammation such as SALM5, HVEM and functional equivalents thereof to provide a beneficial effect, in addition to other effects, by inhibiting the negative regulation of inflammation in the CNS or other immune privileged tissue.

Methods of making and using antibodies are well known in the art. For example, polyclonal antibodies useful in the present invention are generated by immunizing rabbits according to standard immunological techniques well-known in the art (see, e.g., Harlow et al., 1988, In: Antibodies, A Laboratory Manual, Cold Spring Harbor, N.Y.). Such techniques include immunizing an animal with a chimeric protein comprising a portion of another protein such as a maltose binding protein or glutathione (GSH) tag polypeptide portion, and/or a moiety such that the antigenic protein of interest is rendered immunogenic (e.g., an antigen of interest conjugated with keyhole limpet hemocyanin, KLH) and a portion comprising the respective antigenic protein amino acid residues. The chimeric proteins are produced by cloning the appropriate nucleic acids encoding the marker protein into a plasmid vector suitable for this purpose, such as but not limited to, pMAL-2 or pCMX.

However, the invention should not be construed as being limited solely to methods and compositions including these antibodies or to these portions of the antigens. Rather, the invention should be construed to include other antibodies, as that term is defined elsewhere herein, to antigens, or portions thereof. Further, the present invention should be construed to encompass antibodies, inter alia, that bind to the specific antigens of interest, and they are able to bind the antigen present on Western blots, in solution in enzyme linked immunoassays, in fluorescence activated cells sorting (FACS) assays, in magnetic-activated cell sorting (MACS) assays, and in immunofluorescence microscopy of a cell transiently transfected with a nucleic acid encoding at least a portion of the antigenic protein, for example.

One skilled in the art would appreciate, based upon the disclosure provided herein, that the antibody can specifically bind with any portion of the antigen and the full-length protein can be used to generate antibodies specific therefor. However, the present invention is not limited to using the full-length protein as an immunogen. Rather, the present invention includes using an immunogenic portion of the protein to produce an antibody that specifically binds with a specific antigen. That is, the invention includes immunizing an animal using an immunogenic portion, or antigenic determinant, of the antigen.

Once armed with the sequence of a specific antigen of interest and the detailed analysis localizing the various conserved and non-conserved domains of the protein, the skilled artisan would understand, based upon the disclosure provided herein, how to obtain antibodies specific for the various portions of the antigen using methods well-known in the art or to be developed.

The skilled artisan would appreciate, based upon the disclosure provided herein, that the present invention includes use of a single antibody recognizing a single antigenic epitope but that the invention is not limited to use of a single antibody. Instead, the invention encompasses use of at least one antibody where the antibodies can be directed to the same or different antigenic protein epitopes.

The generation of polyclonal antibodies is accomplished by inoculating the desired animal with the antigen and isolating antibodies which specifically bind the antigen therefrom using standard antibody production methods such as those described in, for example, Harlow et al. (1988, In: Antibodies, A Laboratory Manual, Cold Spring Harbor, N.Y.).

Monoclonal antibodies directed against full length or peptide fragments of a protein or peptide may be prepared using any well-known monoclonal antibody preparation procedures, such as those described, for example, in Harlow et al. (1988, In: Antibodies, A Laboratory Manual, Cold Spring Harbor, N.Y.) and in Tuszynski et al. (1988, Blood, 72: 109-115). Quantities of the desired peptide may also be synthesized using chemical synthesis technology. Alternatively, DNA encoding the desired peptide may be cloned and expressed from an appropriate promoter sequence in cells suitable for the generation of large quantities of peptide. Monoclonal antibodies directed against the peptide are generated from mice immunized with the peptide using standard procedures as referenced herein.

Nucleic acid encoding the monoclonal antibody obtained using the procedures described herein may be cloned and sequenced using technology which is available in the art, and is described, for example, in Wright et al. (1992, Critical Rev. Immunol. 12: 125-168), and the references cited therein. Further, the antibody of the invention may be “humanized” using the technology described in, for example, Wright et al., and in the references cited therein, and in Gu et al. (1997, Thrombosis and Hematocyst 77: 755-759), and other methods of humanizing antibodies well-known in the art or to be developed.

The present invention also includes the use of humanized antibodies specifically reactive with epitopes of an antigen of interest. The humanized antibodies of the invention have a human framework and have one or more complementarity determining regions (CDRs) from an antibody, typically a mouse antibody, specifically reactive with an antigen of interest. When the antibody used in the invention is humanized, the antibody may be generated as described in Queen, et al. (U.S. Pat. No. 6,180,370), Wright et al., (supra) and in the references cited therein, or in Gu et al. (1997, Thrombosis and Hematocyst 77(4): 755-759). The method disclosed in Queen et al. is directed in part toward designing humanized immunoglobulins that are produced by expressing recombinant DNA segments encoding the heavy and light chain complementarity determining regions (CDRs) from a donor immunoglobulin capable of binding to a desired antigen, such as an epitope on an antigen of interest, attached to DNA segments encoding acceptor human framework regions. Generally speaking, the invention in the Queen patent has applicability toward the design of substantially any humanized immunoglobulin. Queen explains that the DNA segments will typically include an expression control DNA sequence operably linked to the humanized immunoglobulin coding sequences, including naturally-associated or heterologous promoter regions. The expression control sequences can be eukaryotic promoter systems in vectors capable of transforming or transfecting eukaryotic host cells or the expression control sequences can be prokaryotic promoter systems in vectors capable of transforming or transfecting prokaryotic host cells. Once the vector has been incorporated into the appropriate host, the host is maintained under conditions suitable for high level expression of the introduced nucleotide sequences and as desired the collection and purification of the humanized light chains, heavy chains, light/heavy chain dimers or intact antibodies, binding fragments or other immunoglobulin forms may follow (Beychok, Cells of Immunoglobulin Synthesis, Academic Press, New York, (1979), which is incorporated herein by reference).

The invention also includes functional equivalents of the antibodies described herein. Functional equivalents have binding characteristics comparable to those of the antibodies, and include, for example, hybridized and single chain antibodies, as well as fragments thereof. Methods of producing such functional equivalents are disclosed in PCT Application WO 93/21319 and PCT Application WO 89/09622.

Functional equivalents include polypeptides with amino acid sequences substantially the same as the amino acid sequence of the variable or hypervariable regions of the antibodies. “Substantially the same” amino acid sequence is defined herein as a sequence with at least 70%, preferably at least about 80%, more preferably at least about 90%, even more preferably at least about 95%, and most preferably at least 99% homology to another amino acid sequence (or any integer in between 70 and 99), as determined by the FASTA search method in accordance with Pearson and Lipman, 1988 Proc. Nat'l. Acad. Sci. USA 85: 2444-2448. Chimeric or other hybrid antibodies have constant regions derived substantially or exclusively from human antibody constant regions and variable regions derived substantially or exclusively from the sequence of the variable region of a monoclonal antibody from each stable hybridoma.

Single chain antibodies (scFv) or Fv fragments are polypeptides that consist of the variable region of the heavy chain of the antibody linked to the variable region of the light chain, with or without an interconnecting linker. Thus, the Fv comprises an antibody combining site.

Functional equivalents of the antibodies of the invention further include fragments of antibodies that have the same, or substantially the same, binding characteristics to those of the whole antibody. Such fragments may contain one or both Fab fragments or the F(ab')₂ fragment. The antibody fragments contain all six complement determining regions of the whole antibody, although fragments containing fewer than all of such regions, such as three, four or five complement determining regions, are also functional. The functional equivalents are members of the IgG immunoglobulin class and subclasses thereof, but may be or may combine with any one of the following immunoglobulin classes: IgM, IgA, IgD, or IgE, and subclasses thereof. Heavy chains of various subclasses, such as the IgG subclasses, are responsible for different effector functions and thus, by choosing the desired heavy chain constant region, hybrid antibodies with desired effector function are produced. Exemplary constant regions are gamma 1 (IgG1), gamma 2 (IgG2), gamma 3 (IgG3), and gamma 4 (IgG4). The light chain constant region can be of the kappa or lambda type.

The immunoglobulins of the present invention can be monovalent, divalent or polyvalent. Monovalent immunoglobulins are dimers (HL) formed of a hybrid heavy chain associated through disulfide bridges with a hybrid light chain. Divalent immunoglobulins are tetramers (H₂L₂) formed of two dimers associated through at least one disulfide bridge.

Inhibitory Polypeptides

In another embodiment, SALM5-HVEM antagonists are polypeptides that bind to SALM5 or HVEM. SALM5-binding or HVEM-binding polypeptides can be used to reduce or inhibit the binding of SALM5 to HVEM. Methods of producing polypeptides are well known and within the ability of one of ordinary skill in the art and are described in more detail elsewhere herein.

In some embodiments the polypeptides are soluble fragments of full length SALM5 or HVEM polypeptides. As used herein, a fragment of SALM5 or HVEM refers to any subset of the polypeptide that is a shorter polypeptide of the full length protein. Soluble fragments generally lack some or all of the intracellular and/or transmembrane domains. In some embodiments, soluble fragments of SALM5 or HVEM include the entire extracellular domains of these proteins. In other embodiments, the soluble fragments of SALM5 or HVEM include fragments of the extracellular domains of these proteins. In other embodiments, useful soluble fragments of SALM5 include the LRR domain of SALM5 and useful soluble fragments of HVEM include the CRD1 domain of HVEM.

The polypeptide antagonists can be derived from any species of origin. In a preferred embodiment the polypeptide antagonists are of human origin.

The polypeptides disclosed herein include variant polypeptides. As used herein, a “variant” polypeptide contains at least one amino acid sequence alteration as compared to the amino acid sequence of the corresponding wild-type polypeptide. An amino acid sequence alteration can be, for example, a substitution, a deletion, or an insertion of one or more amino acids.

A variant SALM5-HVEM antagonistic polypeptide can have any combination of amino acid substitutions, deletions or insertions. In one embodiment, isolated SALM5-HVEM antagonistic variant polypeptides have an integer number of amino acid alterations such that their amino acid sequence shares at least 60, 70, 80, 85, 90, 95, 97, 98, 99, 99.5 or 100% identity with an amino acid sequence of a corresponding wild type amino acid sequence. In a preferred embodiment, SALM5-HVEM antagonistic variant polypeptides have an amino acid sequence sharing at least 60, 70, 80, 85, 90, 95, 97, 98, 99, 99.5 or 100% identity with the amino acid sequence of a corresponding wild type polypeptide.

Percent sequence identity can be calculated using computer programs or direct sequence comparison. Preferred computer program methods to determine identity between two sequences include, but are not limited to, the GCG program package, FASTA, BLASTP, and TBLASTN (see, e.g., D. W. Mount, 2001, Bioinformatics: Sequence and Genome Analysis, Cold Spring Harbor Laboratory Press, Cold Spring Harbor, N.Y.). The BLASTP and TBLASTN programs are publicly available from NCBI and other sources. The well-known Smith Waterman algorithm may also be used to determine identity.

Exemplary parameters for amino acid sequence comparison include the following: 1) algorithm from Needleman and Wunsch (J. Mol. Biol., 48: 443-453 (1970)); 2) BLOSSUM62 comparison matrix from Hentikoff and Hentikoff (Proc. Natl. Acad. Sci. U.S.A., 89: 10915-10919 (1992)) 3) gap penalty=12; and 4) gap length penalty=4. A program useful with these parameters is publicly available as the “gap” program (Genetics Computer Group, Madison, Wis.). The aforementioned parameters are the default parameters for polypeptide comparisons (with no penalty for end gaps).

Alternatively, polypeptide sequence identity can be calculated using the following equation: % identity=(the number of identical residues)/(alignment length in amino acid residues)*100. For this calculation, alignment length includes internal gaps but does not include terminal gaps.

Amino acid substitutions in SALM5-HVEM antagonistic variant polypeptides may be “conservative” or “non-conservative”. As used herein, “conservative” amino acid substitutions are substitutions wherein the substituted amino acid has similar structural or chemical properties, and “non-conservative” amino acid substitutions are those in which the charge, hydrophobicity, or bulk of the substituted amino acid is significantly altered. Non-conservative substitutions will differ more significantly in their effect on maintaining (a) the structure of the peptide backbone in the area of the substitution, for example, as a sheet or helical conformation, (b) the charge or hydrophobicity of the molecule at the target site, or (c) the bulk of the side chain. Examples of conservative amino acid substitutions include those in which the substitution is within one of the five following groups: 1) small aliphatic, nonpolar or slightly polar residues (Ala, Ser, Thr, Pro, Gly); 2) polar, negatively charged residues and their amides (Asp, Asn, Glu, Gln); polar, positively charged residues (His, Arg, Lys); large aliphatic, nonpolar residues (Met, Leu, Ile, Val, Cys); and large aromatic resides (Phe, Tyr, Trp). Examples of non-conservative amino acid substitutions are those where 1) a hydrophilic residue, e.g., seryl or threonyl, is substituted for (or by) a hydrophobic residue, e.g., leucyl, isoleucyl, phenylalanyl, valyl, or alanyl; 2) a cysteine or proline is substituted for (or by) any other residue; 3) a residue having an electropositive side chain, e.g., lysyl, arginyl, or histidyl, is substituted for (or by) an electronegative residue, e.g., glutamyl or aspartyl; or 4) a residue having a bulky side chain, e.g., phenylalanine, is substituted for (or by) a residue that does not have a side chain, e.g., glycine.

SALM5-HVEM antagonistic variant polypeptides may be modified by chemical moieties that may be present in polypeptides in a normal cellular environment, for example, phosphorylation, methylation, amidation, sulfation, acylation, glycosylation, sumoylation and ubiquitylation. SALM5-HVEM antagonistic variant polypeptides may also be modified with a label capable of providing a detectable signal, either directly or indirectly, including, but not limited to, radioisotopes and fluorescent compounds.

SALM5-HVEM antagonistic variant polypeptides may also be modified by chemical moieties that are not normally added to polypeptides in a cellular environment. Such modifications may be introduced into the molecule by reacting targeted amino acid residues of the polypeptide with an organic derivatizing agent that is capable of reacting with selected side chains or terminal residues. Another modification is cyclization of the protein.

Small Molecules and Other Antagonists

It will be appreciated that additional bioactive agents may be screened for SALM5-HVEM antagonistic activity. In one embodiment, candidate bioactive agents are screened for their ability to reduce binding of SALM5 to HVEM. In another embodiment, candidate bioactive agents are screened for their ability to reduce activation of either SALM5 or HVEM. The assays preferably utilize human SALM5 and human HVEM proteins, although other SALM5 and HVEM proteins may also be used.

The term “candidate bioactive agent” as used herein describes any molecule, e.g., protein, small organic molecule, carbohydrates (including polysaccharides), polynucleotide, lipids, etc. Generally a plurality of assay mixtures are run in parallel with different agent concentrations to obtain a differential response to the various concentrations. Typically, one of these concentrations serves as a negative control, i.e., at zero concentration or below the level of detection. In addition, positive controls, i.e. the use of agents known to bind SALM5 or HVEM may be used.

Candidate agents encompass numerous chemical classes, though typically they are organic molecules, preferably small organic compounds having a molecular weight of more than 100 and less than about 2,500 daltons, more preferably between 100 and 2000, more preferably between about 100 and about 1250, more preferably between about 100 and about 1000, more preferably between about 100 and about 750, more preferably between about 200 and about 500 daltons. Candidate agents comprise functional groups necessary for structural interaction with proteins, particularly hydrogen bonding, and typically include at least an amine, carbonyl, hydroxyl or carboxyl group, preferably at least two of the functional chemical groups. The candidate agents often comprise cyclical carbon or heterocyclic structures and/or aromatic or polyaromatic structures substituted with one or more of the above functional groups. Candidate agents are also found among biomolecules including peptides, saccharides, fatty acids, steroids, purines, pyrimidines, derivatives, structural analogs or combinations thereof. Particularly preferred are peptides, e.g., peptidomimetics. Peptidomimetics can be made as described, e.g., in WO 98156401.

Candidate agents are obtained from a wide variety of sources including libraries of synthetic or natural compounds. For example, numerous means are available for random and directed synthesis of a wide variety of organic compounds and biomolecules, including expression of randomized oligonucleotides. Alternatively, libraries of natural compounds in the form of bacterial, fungal, plant and animal extracts are available or readily produced. Additionally, natural or synthetically produced libraries and compounds are readily modified through conventional chemical, physical and biochemical means. Known pharmacological agents may be subjected to directed or random chemical modifications, such as acylation, alkylation, esterification, amidification to produce structural analogs. In a preferred embodiment, the candidate bioactive agents are organic chemical moieties or small molecule chemical compositions, a wide variety of which are available in the art

Antagonists that Reduce or Inhibit the Expression of SALM5 or HVEM

In one embodiment SALM5-HVEM antagonists reduce or inhibit the expression of SALM5 or HVEM. SALM5-HVEM antagonists that reduce or inhibit expression of SALM5 or HVEM include inhibitory nucleic acids, including, but not limited to, ribozymes, triplex-forming oligonucleotides (TFOs), antisense DNA, siRNA, and microRNA specific for nucleic acids encoding SALM5 or HVEM.

Useful inhibitory nucleic acids include those that reduce the expression of RNA encoding SALM5 or HVEM by at least 20%, 30%, 40%, 50%, 60%, 70%, 80%, 90% or 95% compared to controls. Expression of SALM5 or HVEM can be measured by methods well known to those of skill in the art, including northern blotting and quantitative polymerase chain reaction (PCR).

An siRNA polynucleotide is an RNA nucleic acid molecule that interferes with RNA activity that is generally considered to occur via a post-transcriptional gene silencing mechanism. An siRNA polynucleotide preferably comprises a double-stranded RNA (dsRNA) but is not intended to be so limited and may comprise a single-stranded RNA (see, e.g., Martinez et al., 2002 Cell 110: 563-74). The siRNA polynucleotide included in the invention may comprise other naturally occurring, recombinant, or synthetic single-stranded or double-stranded polymers of nucleotides (ribonucleotides or deoxyribonucleotides or a combination of both) and/or nucleotide analogues as provided herein (e.g., an oligonucleotide or polynucleotide or the like, typically in 5′ to 3′ phosphodiester linkage). Accordingly it will be appreciated that certain exemplary sequences disclosed herein as DNA sequences capable of directing the transcription of the siRNA polynucleotides are also intended to describe the corresponding RNA sequences and their complements, given the well-established principles of complementary nucleotide base-pairing.

Preferred siRNA polynucleotides comprise double-stranded polynucleotides of about 18-30 nucleotide base pairs, preferably about 18, about 19, about 20, about 21, about 22, about 23, about 24, about 25, about 26, or about 27 base pairs, and in other preferred embodiments about 19, about 20, about 21, about 22 or about 23 base pairs, or about 27 base pairs, whereby the use of “about” indicates that in certain embodiments and under certain conditions the processive cleavage steps that may give rise to functional siRNA polynucleotides that are capable of interfering with expression of a selected polypeptide may not be absolutely efficient. Hence, siRNA polynucleotides, may include one or more siRNA polynucleotide molecules that may differ (e.g., by nucleotide insertion or deletion) in length by one, two, three, four or more base pairsas a consequence of the variability in processing, in biosynthesis, or in artificial synthesis of the siRNA. The siRNA polynucleotide of the present invention may also comprise a polynucleotide sequence that exhibits variability by differing (e.g., by nucleotide substitution, including transition or transversion) at one, two, three or four nucleotides from a particular sequence. These differences can occur at any of the nucleotide positions of a particular siRNA polynucleotide sequence, depending on the length of the molecule, whether situated in a sense or in an antisense strand of the double-stranded polynucleotide. The nucleotide difference may be found on one strand of a double-stranded polynucleotide, where the complementary nucleotide with which the substitute nucleotide would typically form hydrogen bond base pairing, may not necessarily be correspondingly substituted. In preferred embodiments, the siRNA polynucleotides are homogeneous with respect to a specific nucleotide sequence.

One skilled in the art will readily appreciate that as a result of the degeneracy of the genetic code, many different nucleotide sequences may encode the same polypeptide. That is, an amino acid may be encoded by one of several different codons, and a person skilled in the art can readily determine that while one particular nucleotide sequence may differ from another, the polynucleotides may in fact encode polypeptides with identical amino acid sequences. As such, polynucleotides that vary due to differences in codon usage are specifically contemplated by the present invention.

One skilled in the art will appreciate, based on the disclosure provided herein, that one way to decrease the mRNA and/or protein levels of SALM5, HVEM and functional equivalents thereof in a cell is by reducing or inhibiting expression of the nucleic acid encoding the negative regulator of inflammation of the invention. Thus, the protein level of the regulator in a cell can also be decreased using a molecule or compound that inhibits or reduces gene expression such as, for example, an antisense molecule or a ribozyme.

In a preferred embodiment, the modulating sequence is an antisense nucleic acid sequence which is expressed by a plasmid vector. The antisense expressing vector is used to transfect a mammalian cell or the mammal itself, thereby causing reduced endogenous expression of a desired negative regulator of inflammation of the invention in the cell. However, the invention should not be construed to be limited to inhibiting expression of a regulator by transfection of cells with antisense molecules. Rather, the invention encompasses other methods known in the art for inhibiting expression or activity of a protein in the cell including, but not limited to, the use of a ribozyme, the expression of a non-functional regulator (i.e. transdominant negative mutant) and use of an intracellular antibody.

Antisense molecules and their use for inhibiting gene expression are well known in the art (see, e.g., Cohen, 1989, In: Oligodeoxyribonucleotides, Antisense Inhibitors of Gene Expression, CRC Press). Antisense nucleic acids are DNA or RNA molecules that are complementary, as that term is defined elsewhere herein, to at least a portion of a specific mRNA molecule (Weintraub, 1990, Scientific American 262: 40). In the cell, antisense nucleic acids hybridize to the corresponding mRNA, forming a double-stranded molecule thereby inhibiting the translation of genes.

The use of antisense methods to inhibit the translation of genes is known in the art, and is described, for example, in Marcus-Sakura (1988, Anal. Biochem. 172: 289). Such antisense molecules may be provided to the cell via genetic expression using DNA encoding the antisense molecule as taught by Inoue, 1993, U.S. Pat. No. 5,190,931.

Alternatively, antisense molecules of the invention may be made synthetically and then provided to the cell. Antisense oligomers of between about 10 to about 30, and more preferably about 15 nucleotides, are preferred, since they are easily synthesized and introduced into a target cell. Synthetic antisense molecules contemplated by the invention include oligonucleotide derivatives known in the art which have improved biological activity compared to unmodified oligonucleotides (see U.S. Pat. No. 5,023,243).

Ribozymes and their use for inhibiting gene expression are also well known in the art (see, e.g., Cech et al., 1992, J. Biol. Chem. 267: 17479-17482; Hampel et al., 1989, Biochemistry 28: 4929-4933; Eckstein et al., International Publication No. WO 92/07065; Altman et al., U.S. Pat. No. 5,168,053). Ribozymes are RNA molecules possessing the ability to specifically cleave other single-stranded RNA in a manner analogous to DNA restriction endonucleases. Through the modification of nucleotide sequences encoding these RNAs, molecules can be engineered to recognize specific nucleotide sequences in an RNA molecule and cleave it (Cech, 1988, J. Amer. Med. Assn. 260: 3030). A major advantage of this approach is the fact that ribozymes are sequence-specific.

There are two basic types of ribozymes, namely, tetrahymena-type (Hasselhoff, 1988, Nature 334: 585) and hammerhead-type. Tetrahymena-type ribozymes recognize sequences which are four bases in length, while hammerhead-type ribozymes recognize base sequences 11-18 bases in length. The longer the sequence, the greater the likelihood that the sequence will occur exclusively in the target mRNA species. Consequently, hammerhead-type ribozymes are preferable to tetrahymena-type ribozymes for inactivating specific mRNA species, and 18-base recognition sequences are preferable to shorter recognition sequences which may occur randomly within various unrelated mRNA molecules.

Ribozymes useful for inhibiting the expression of a negative regulator of inflammation of the invention may be designed by incorporating target sequences into the basic ribozyme structure which are complementary to the mRNA sequence of the desired negative regulator of inflammation of the present invention, including but are not limited to, SALM5, HVEM and functional equivalents thereof. Ribozymes targeting the desired negative regulator of inflammation of the invention may be synthesized using commercially available reagents (Applied Biosystems, Inc., Foster City, Calif.) or they may be genetically expressed from DNA encoding them.

### Activating Compositions

As described elsewhere herein, the invention is based on the discovery that activation of SALM5, HVEM, or both as well as their functional equivalents and interactions thereof contributes to the regulation of inflammation in the CNS or other immune privileged tissue. This observation is the first of its kind by providing a direct link between the molecule pathway involving SALM5 and the negative regulation and control of neuroinflammation. As such, the invention provides for a way to therapeutically activate the negative regulation and control of neuroinflammation in the CNS or other immune privileged tissue. This activation of a negative regulator of inflammation in the immune privileged tissue can be beneficial, in addition to other effects, by activating the expression of SALM5, HVEM, or both as well as their functional equivalents and interactions thereof in order to reduce inflammation in the CNS or other immune privilege tissue.

Based on the disclosure herein, the present invention includes a generic concept for activating a negative regulator of inflammation or any component of the signal transduction pathway associated with the interaction between SALM5 and HVEM or functional equivalents thereof. Preferably, the negative regulator of inflammation includes SALM5, HVEM and functional equivalents thereof, whereby activating any one or more of these proteins is associated with reducing inflammation in the CNS or other immune privileged tissue.

In the case of inflammation of the brain, a variety of clinical situations, including mechanical trauma (e.g. traumatic brain injury), ischaemia (e.g. stroke) and infection (e.g. meningitis), autoimmunity, and inflammatory disease, can lead to an inflammatory response within the brain and central nervous system. This inflammatory response is responsible for much of the long term and permanent damage caused to the brain by these conditions. However, most of the permanent damage that occurs is not caused by the triggering event, but rather by the body's subsequent reaction to the insult (secondary injury process). A major aspect of that secondary injury process is the inflammatory reaction that is initiated in response to the primary injury. This inflammatory response is characterized by the release of pro-inflammatory mediators which may contribute to the brain damage through the release of neurotoxic substances. It has been shown that a number cytokines, such as IL-1β, IL-6 and TNFα, are substantially increased following an insult within the brain and that amongst other effects, these cytokines contribute to the activation and proliferation of astrocytes, and the recruitment of neutrophils into the CNS, which leads to further tissue damage. There is clinical evidence that these increases in cytokine levels are associated with cerebral injury and the development of neurological deficits.

In one embodiment, agonists or activators of the invention are able to decrease inflammation in the CNS or other immune privileged tissue. Activation of one or more of SALM5, HVEM and functional equivalents thereof can be accomplished by way of increasing expression of SALM5, HVEM and functional equivalents thereof. In another embodiment, an agonist of the invention can be a nucleic acid molecule encoding SALM5, HVEM and functional equivalents thereof. In another embodiment, an agonist of the invention mimics the biological function of SALM5 with respect to exhibiting a suppressive effect in the CNS or other immune privileged tissue when the agonist binds to HVEM.

In one embodiment, overexpression of one or more of SALM5, HVEM and functional equivalents thereof can reduce tissue inflammation, thereby preventing the harmful effects of inflammation in the CNS or other immune privileged tissue.

In one embodiment, the invention comprises a composition for diminishing inflammation in the CNS or other immune privileged tissue. The composition comprises an activator of any one or more of the following negative regulator of inflammation of the invention: SALM5, HVEM and functional equivalents thereof. The composition comprising the inhibitor includes but is not limited to a nucleic acid encoding one or more of SALM5, HVEM and functional equivalents thereof, a polypeptide of one or more of SALM5, HVEM and functional equivalents thereof, a small molecule, an activating antibody, and the likes.

Agonists or Activators of SALM5-HVEM Interaction

SALM5-HVEM agonists that induce or increase the binding of SALM5 to HVEM can induce the suppressive effects of SALM5 on inflammation. Accordingly, SALM5-HVEM agonists of the invention can suppress inflammation in the CNS or other immune privileged tissue. In one embodiment, SALM5-HVEM agonists of the invention induce the binding of SALM5-HVEM by inducing the binding of the LRR domain of SALM5 to the CRD1 domain of HVEM. The increase in binding between SALM5 and HVEM can be by more than 50%, 40%, 30%, 20%, 10%, 5% or less, as compared to controls. SALM5-HVEM agonists that increase or induce the binding of SALM5 to HVEM include activating antibodies and antibody fragments that bind HVEM or SALM5, SALM5 or HVEM activating polypeptides including soluble fragments of SALM5 or HVEM, and small organic compounds.

In one embodiment, SALM5-HVEM agonists that bind to SALM5 or HVEM increase or induce the interaction between SALM5 and HVEM by at least 20%, more preferably by at least 30%, more preferably by at least 40%, 50%, 60%, 70%, 80%, 90%, 95%, 96%, 97%, 98%, 99% or more.

In one embodiment, SALM5-HVEM agonists that are capable of binding to SALM5 or HVEM increase SALM5 or HVEM activity in a cell expressing SALM5 or HVEM on its surface. In some embodiments SALM5-HVEM agonists are capable of increasing or inducing one or more activities of SALM5 or HVEM in a cell expressing SALM5 or HVEM on its surface. In some embodiments, the cell expressing HVEM is a lymphocyte, a T cell, a CD4+ T cell, a CD8+ T cell, a Th1 cell, a B cell, a plasma cell, a macrophage, or an NK cell. In preferred embodiments, the cell expressing HVEM is a myeloid cell. In some embodiment, the cell expressing SALM5 is a cell of the CNS, preferably a neuronal cell. SALM5-HVEM agonists that bind to SALM5 or HVEM induce or increase one or more SALM5 or HVEM activities by at least 20%, more preferably by at least 30%, more preferably by at least 40%, 50%, 60%, 70%, 80%, 90%, 95%, 96%, 97%, 98%, 99% or more.

Activating Antibodies

In one embodiment, SALM5-HVEM agonists are antibodies. Antibodies or antibody fragments that specifically bind to SALM5 or HVEM can be used to increase or induce the binding of SALM5 to HVEM. Methods of producing antibodies are well known and within the ability of one of ordinary skill in the art and are described in more detail elsewhere herein.

The antibodies disclosed herein specifically bind to a SALM5 or an HVEM protein and are capable of increasing or inducing the binding of SALM5 to HVEM. These antibodies are defined as “activating,” “function-inducing” or “agonistic” antibodies. In preferred embodiments the agonistic antibodies specifically bind to a portion of the extracellular domain of SALM5 or HVEM. In other embodiments, the agonistic antibodies specifically bind to the LRR domain of SALM5 or the CRD1 domain of HVEM.

As will be understood by one skilled in the art, any antibody that can recognize and bind to an antigen of interest is useful in the present invention. That is, the antibody can activate a negative regulator of inflammation of the invention such as SALM5, HVEM and functional equivalents thereof to provide a beneficial effect, in addition to other effects, by inducing the negative regulation of inflammation in the CNS or other immune privileged tissue.

Methods of making and using antibodies are well known in the art. For example, polyclonal antibodies useful in the present invention are generated by immunizing rabbits according to standard immunological techniques well-known in the art (see, e.g., Harlow et al., 1988, In: Antibodies, A Laboratory Manual, Cold Spring Harbor, N.Y.). Such techniques include immunizing an animal with a chimeric protein comprising a portion of another protein such as a maltose binding protein or glutathione (GSH) tag polypeptide portion, and/or a moiety such that the antigenic protein of interest is rendered immunogenic (e.g., an antigen of interest conjugated with keyhole limpet hemocyanin, KLH) and a portion comprising the respective antigenic protein amino acid residues. The chimeric proteins are produced by cloning the appropriate nucleic acids encoding the marker protein into a plasmid vector suitable for this purpose, such as but not limited to, pMAL-2 or pCMX.

However, the invention should not be construed as being limited solely to methods and compositions including these antibodies or to these portions of the antigens.

Rather, the invention should be construed to include other antibodies, as that term is defined elsewhere herein, to antigens, or portions thereof. Further, the present invention should be construed to encompass antibodies, inter alia, bind to the specific antigens of interest, and they are able to bind the antigen present on Western blots, in solution in enzyme linked immunoassays, in fluorescence activated cells sorting (FACS) assays, in magnetic-activated cell sorting (MACS) assays, and in immunofluorescence microscopy of a cell transiently transfected with a nucleic acid encoding at least a portion of the antigenic protein, for example.

One skilled in the art would appreciate, based upon the disclosure provided herein, that the antibody can specifically bind with any portion of the antigen and the full-length protein can be used to generate antibodies specific therefor. However, the present invention is not limited to using the full-length protein as an immunogen. Rather, the present invention includes using an immunogenic portion of the protein to produce an antibody that specifically binds with a specific antigen. That is, the invention includes immunizing an animal using an immunogenic portion, or antigenic determinant, of the antigen.

Once armed with the sequence of a specific antigen of interest and the detailed analysis localizing the various conserved and non-conserved domains of the protein, the skilled artisan would understand, based upon the disclosure provided herein, how to obtain antibodies specific for the various portions of the antigen using methods well-known in the art or to be developed.

The skilled artisan would appreciate, based upon the disclosure provided herein, that the present invention includes use of a single antibody recognizing a single antigenic epitope but that the invention is not limited to use of a single antibody. Instead, the invention encompasses use of at least one antibody where the antibodies can be directed to the same or different antigenic protein epitopes.

The generation of polyclonal antibodies is accomplished by inoculating the desired animal with the antigen and isolating antibodies which specifically bind the antigen therefrom using standard antibody production methods such as those described in, for example, Harlow et al. (1988, In: Antibodies, A Laboratory Manual, Cold Spring Harbor, N.Y.).

Monoclonal antibodies directed against full length or peptide fragments of a protein or peptide may be prepared using any well-known monoclonal antibody preparation procedures, such as those described, for example, in Harlow et al. (1988, In: Antibodies, A Laboratory Manual, Cold Spring Harbor, N.Y.) and in Tuszynski et al. (1988, Blood, 72: 109-115). Quantities of the desired peptide may also be synthesized using chemical synthesis technology. Alternatively, DNA encoding the desired peptide may be cloned and expressed from an appropriate promoter sequence in cells suitable for the generation of large quantities of peptide. Monoclonal antibodies directed against the peptide are generated from mice immunized with the peptide using standard procedures as referenced herein.

Nucleic acid encoding the monoclonal antibody obtained using the procedures described herein may be cloned and sequenced using technology which is available in the art, and is described, for example, in Wright et al. (1992, Critical Rev. Immunol. 12: 125-168), and the references cited therein. Further, the antibody of the invention may be “humanized” using the technology described in, for example, Wright et al., and in the references cited therein, and in Gu et al. (1997, Thrombosis and Hematocyst 77: 755-759), and other methods of humanizing antibodies well-known in the art or to be developed.

The present invention also includes the use of humanized antibodies specifically reactive with epitopes of an antigen of interest. The humanized antibodies of the invention have a human framework and have one or more complementarity determining regions (CDRs) from an antibody, typically a mouse antibody, specifically reactive with an antigen of interest. When the antibody used in the invention is humanized, the antibody may be generated as described in Queen, et al. (U.S. Pat. No. 6,180,370), Wright et al., (supra) and in the references cited therein, or in Gu et al. (1997, Thrombosis and Hematocyst 77(4): 755-759). The method disclosed in Queen et al. is directed in part toward designing humanized immunoglobulins that are produced by expressing recombinant DNA segments encoding the heavy and light chain complementarity determining regions (CDRs) from a donor immunoglobulin capable of binding to a desired antigen, such as an epitope on an antigen of interest, attached to DNA segments encoding acceptor human framework regions. Generally speaking, the invention in the Queen patent has applicability toward the design of substantially any humanized immunoglobulin. Queen explains that the DNA segments will typically include an expression control DNA sequence operably linked to the humanized immunoglobulin coding sequences, including naturally-associated or heterologous promoter regions. The expression control sequences can be eukaryotic promoter systems in vectors capable of transforming or transfecting eukaryotic host cells or the expression control sequences can be prokaryotic promoter systems in vectors capable of transforming or transfecting prokaryotic host cells. Once the vector has been incorporated into the appropriate host, the host is maintained under conditions suitable for high level expression of the introduced nucleotide sequences and as desired the collection and purification of the humanized light chains, heavy chains, light/heavy chain dimers or intact antibodies, binding fragments or other immunoglobulin forms may follow (Beychok, Cells of Immunoglobulin Synthesis, Academic Press, New York, (1979), which is incorporated herein by reference). The invention also includes functional equivalents of the antibodies described herein. Functional equivalents have binding characteristics comparable to those of the antibodies, and include, for example, hybridized and single chain antibodies, as well as fragments thereof. Methods of producing such functional equivalents are disclosed in PCT Application WO 93/21319 and PCT Application WO 89/09622.

Functional equivalents include polypeptides with amino acid sequences substantially the same as the amino acid sequence of the variable or hypervariable regions of the antibodies. “Substantially the same” amino acid sequence is defined herein as a sequence with at least 70%, preferably at least about 80%, more preferably at least about 90%, even more preferably at least about 95%, and most preferably at least 99% homology to another amino acid sequence (or any integer in between 70 and 99), as determined by the FASTA search method in accordance with Pearson and Lipman, 1988 Proc. Nat'l. Acad. Sci. USA 85: 2444-2448. Chimeric or other hybrid antibodies have constant regions derived substantially or exclusively from human antibody constant regions and variable regions derived substantially or exclusively from the sequence of the variable region of a monoclonal antibody from each stable hybridoma.

Single chain antibodies (scFv) or Fv fragments are polypeptides that consist of the variable region of the heavy chain of the antibody linked to the variable region of the light chain, with or without an interconnecting linker. Thus, the Fv comprises an antibody combining site.

Functional equivalents of the antibodies of the invention further include fragments of antibodies that have the same, or substantially the same, binding characteristics to those of the whole antibody. Such fragments may contain one or both Fab fragments or the F(ab')₂ fragment. The antibody fragments contain all six complement determining regions of the whole antibody, although fragments containing fewer than all of such regions, such as three, four or five complement determining regions, are also functional. The functional equivalents are members of the IgG immunoglobulin class and subclasses thereof, but may be or may combine with any one of the following immunoglobulin classes: IgM, IgA, IgD, or IgE, and subclasses thereof. Heavy chains of various subclasses, such as the IgG subclasses, are responsible for different effector functions and thus, by choosing the desired heavy chain constant region, hybrid antibodies with desired effector function are produced. Exemplary constant regions are gamma 1 (IgG1), gamma 2 (IgG2), gamma 3 (IgG3), and gamma 4 (IgG4). The light chain constant region can be of the kappa or lambda type.

The immunoglobulins of the present invention can be monovalent, divalent or polyvalent. Monovalent immunoglobulins are dimers (HL) formed of a hybrid heavy chain associated through disulfide bridges with a hybrid light chain. Divalent immunoglobulins are tetramers (H₂L₂) formed of two dimers associated through at least one disulfide bridge.

Activating Polypeptides

In another embodiment, SALM5-HVEM agonists are polypeptides that bind to SALM5 or HVEM. SALM5- or HVEM-binding polypeptides can be used to increase or induce the binding of SALM5 to HVEM. Methods of producing polypeptides are well known and within the ability of one of ordinary skill in the art and are described in more detail elsewhere herein.

In some embodiments the polypeptides are soluble fragments of full length SALM5 or HVEM polypeptides as well as ligands thereof. As used herein, a fragment of SALM5 or HVEM refers to any subset of the polypeptide that is a shorter polypeptide of the full length protein. Soluble fragments generally lack some or all of the intracellular and/or transmembrane domains. In some embodiments, soluble fragments of SALM5 or HVEM as well as ligands thereof include the entire extracellular domains of these proteins. In other embodiments, the soluble fragments of SALM5 or HVEM include fragments of the extracellular domains of these proteins. In other embodiments, useful soluble fragments of SALM5 include the LRR domain of SALM5 and useful soluble fragments of HVEM include the CRD1 domain of HVEM.

The polypeptide SALM5-HVEM agonists can be derived from any species of origin. In a preferred embodiment the polypeptide SALM5-HVEM agonists are of human origin.

The polypeptides disclosed herein include variant polypeptides. As used herein, a “variant” polypeptide contains at least one amino acid sequence alteration as compared to the amino acid sequence of the corresponding wild-type polypeptide. An amino acid sequence alteration can be, for example, a substitution, a deletion, or an insertion of one or more amino acids.

A variant SALM5-HVEM agonistic polypeptide can have any combination of amino acid substitutions, deletions or insertions. In one embodiment, isolated SALM5-HVEM agonistic variant polypeptides have an integer number of amino acid alterations such that their amino acid sequence shares at least 60, 70, 80, 85, 90, 95, 97, 98, 99, 99.5 or 100% identity with an amino acid sequence of a corresponding wild type amino acid sequence. In a preferred embodiment, SALM5-HVEM agonistic variant polypeptides have an amino acid sequence sharing at least 60, 70, 80, 85, 90, 95, 97, 98, 99, 99.5 or 100% identity with the amino acid sequence of a corresponding wild type polypeptide.

Percent sequence identity can be calculated using computer programs or direct sequence comparison. Preferred computer program methods to determine identity between two sequences include, but are not limited to, the GCG program package, FASTA, BLASTP, and TBLASTN (see, e.g., D. W. Mount, 2001, Bioinformatics: Sequence and Genome Analysis, Cold Spring Harbor Laboratory Press, Cold Spring Harbor, N.Y.). The BLASTP and TBLASTN programs are publicly available from NCBI and other sources. The well-known Smith Waterman algorithm may also be used to determine identity.

Exemplary parameters for amino acid sequence comparison include the following: 1) algorithm from Needleman and Wunsch (J. Mol. Biol., 48: 443-453 (1970)); 2) BLOSSUM62 comparison matrix from Hentikoff and Hentikoff (Proc. Natl. Acad. Sci. U.S.A., 89: 10915-10919 (1992)) 3) gap penalty=12; and 4) gap length penalty=4. A program useful with these parameters is publicly available as the “gap” program (Genetics Computer Group, Madison, Wis.). The aforementioned parameters are the default parameters for polypeptide comparisons (with no penalty for end gaps).

Alternatively, polypeptide sequence identity can be calculated using the following equation: % identity=(the number of identical residues)/(alignment length in amino acid residues)*100. For this calculation, alignment length includes internal gaps but does not include terminal gaps.

Amino acid substitutions in SALM5-HVEM agonistic variant polypeptides may be “conservative” or “non-conservative”. As used herein, “conservative” amino acid substitutions are substitutions wherein the substituted amino acid has similar structural or chemical properties, and “non-conservative” amino acid substitutions are those in which the charge, hydrophobicity, or bulk of the substituted amino acid is significantly altered. Non-conservative substitutions will differ more significantly in their effect on maintaining (a) the structure of the peptide backbone in the area of the substitution, for example, as a sheet or helical conformation, (b) the charge or hydrophobicity of the molecule at the target site, or (c) the bulk of the side chain.

Examples of conservative amino acid substitutions include those in which the substitution is within one of the five following groups: 1) small aliphatic, nonpolar or slightly polar residues (Ala, Ser, Thr, Pro, Gly); 2) polar, negatively charged residues and their amides (Asp, Asn, Glu, Gln); polar, positively charged residues (His, Arg, Lys); large aliphatic, nonpolar residues (Met, Leu, Ile, Val, Cys); and large aromatic resides (Phe, Tyr, Trp). Examples of non-conservative amino acid substitutions are those where 1) a hydrophilic residue, e.g., seryl or threonyl, is substituted for (or by) a hydrophobic residue, e.g., leucyl, isoleucyl, phenylalanyl, valyl, or alanyl; 2) a cysteine or proline is substituted for (or by) any other residue; 3) a residue having an electropositive side chain, e.g., lysyl, arginyl, or histidyl, is substituted for (or by) an electronegative residue, e.g., glutamyl or aspartyl; or 4) a residue having a bulky side chain, e.g., phenylalanine, is substituted for (or by) a residue that does not have a side chain, e.g., glycine. SALM5-HVEM agonistic variant polypeptides may be modified by chemical moieties that may be present in polypeptides in a normal cellular environment, for example, phosphorylation, methylation, amidation, sulfation, acylation, glycosylation, sumoylation and ubiquitylation. SALM5-HVEM agonistic variant polypeptides may also be modified with a label capable of providing a detectable signal, either directly or indirectly, including, but not limited to, radioisotopes and fluorescent compounds.

SALM5-HVEM agonistic variant polypeptides may also be modified by chemical moieties that are not normally added to polypeptides in a cellular environment. Such modifications may be introduced into the molecule by reacting targeted amino acid residues of the polypeptide with an organic derivatizing agent that is capable of reacting with selected side chains or terminal residues. Another modification is cyclization of the protein.

Small Molecules and Other Agonists

It will be appreciated that additional bioactive agents may be screened for SALM5-HVEM agonistic activity. In one embodiment, candidate bioactive agents are screened for their ability to increase binding of SALM5 to HVEM. In another embodiment, candidate bioactive agents are screened for their ability to increase activation of either SALM5 or HVEM. The assays preferably utilize human SALM5 and human HVEM proteins, although other SALM5 and HVEM proteins may also be used.

The term “candidate bioactive agent” as used herein describes any molecule, e.g., protein, small organic molecule, carbohydrates (including polysaccharides), polynucleotide, lipids, etc. Generally a plurality of assay mixtures are run in parallel with different agent concentrations to obtain a differential response to the various concentrations. Typically, one of these concentrations serves as a negative control, i.e., at zero concentration or below the level of detection. In addition, positive controls, i.e. the use of agents known to bind SALM5 or HVEM may be used.

Candidate agents encompass numerous chemical classes, though typically they are organic molecules, preferably small organic compounds having a molecular weight of more than 100 and less than about 2,500 daltons, more preferably between 100 and 2000, more preferably between about 100 and about 1250, more preferably between about 100 and about 1000, more preferably between about 100 and about 750, more preferably between about 200 and about 500 daltons. Candidate agents comprise functional groups necessary for structural interaction with proteins, particularly hydrogen bonding, and typically include at least an amine, carbonyl, hydroxyl or carboxyl group, preferably at least two of the functional chemical groups. The candidate agents often comprise cyclical carbon or heterocyclic structures and/or aromatic or polyaromatic structures substituted with one or more of the above functional groups. Candidate agents are also found among biomolecules including peptides, saccharides, fatty acids, steroids, purines, pyrimidines, derivatives, structural analogs or combinations thereof. Particularly preferred are peptides, e.g., peptidomimetics. Peptidomimetics can be made as described, e.g., in WO 98156401.

Candidate agents are obtained from a wide variety of sources including libraries of synthetic or natural compounds. For example, numerous means are available for random and directed synthesis of a wide variety of organic compounds and biomolecules, including expression of randomized oligonucleotides. Alternatively, libraries of natural compounds in the form of bacterial, fungal, plant and animal extracts are available or readily produced. Additionally, natural or synthetically produced libraries and compounds are readily modified through conventional chemical, physical and biochemical means. Known pharmacological agents may be subjected to directed or random chemical modifications, such as acylation, alkylation, esterification, amidification to produce structural analogs. In a preferred embodiment, the candidate bioactive agents are organic chemical moieties or small molecule chemical compositions, a wide variety of which are available in the art

Agonists that Increase or Induce the Expression of SALM5 or HVEM

In one embodiment SALM5-HVEM agonists increase or induce the expression of SALM5 or HVEM by inhibiting negative regulators of SALM5 or HVEM, and their equivalents. SALM5-HVEM agonists that increase or induce expression of SALM5 or HVEM, and their equivalents include inhibitory nucleic acids, including, but not limited to, ribozymes, triplex-forming oligonucleotides (TFOs), antisense DNA, siRNA, and microRNA specific for nucleic acids encoding SALM5 or HVEM.

Useful inhibitory nucleic acids that inhibit negative regulators of SALM5 or HVEM include those that increase the expression of RNA encoding SALM5 or HVEM by at least 20%, 30%, 40%, 50%, 60%, 70%, 80%, 90% or 95% compared to controls. Expression of SALM5 or HVEM can be measured by methods well known to those of skill in the art, including northern blotting and quantitative polymerase chain reaction (PCR).

An siRNA polynucleotide is an RNA nucleic acid molecule that interferes with RNA activity that is generally considered to occur via a post-transcriptional gene silencing mechanism. An siRNA polynucleotide preferably comprises a double-stranded RNA (dsRNA) but is not intended to be so limited and may comprise a single-stranded RNA (see, e.g., Martinez et al., 2002 Cell 110: 563-74). The siRNA polynucleotide included in the invention may comprise other naturally occurring, recombinant, or synthetic single-stranded or double-stranded polymers of nucleotides (ribonucleotides or deoxyribonucleotides or a combination of both) and/or nucleotide analogues as provided herein (e.g., an oligonucleotide or polynucleotide or the like, typically in 5′ to 3′ phosphodiester linkage). Accordingly it will be appreciated that certain exemplary sequences disclosed herein as DNA sequences capable of directing the transcription of the siRNA polynucleotides are also intended to describe the corresponding RNA sequences and their complements, given the well-established principles of complementary nucleotide base-pairing.

Preferred siRNA polynucleotides comprise double-stranded polynucleotides of about 18-30 nucleotide base pairs, preferably about 18, about 19, about 20, about 21, about 22, about 23, about 24, about 25, about 26, or about 27 base pairs, and in other preferred embodiments about 19, about 20, about 21, about 22 or about 23 base pairs, or about 27 base pairs, whereby the use of “about” indicates that in certain embodiments and under certain conditions the processive cleavage steps that may give rise to functional siRNA polynucleotides that are capable of interfering with expression of a selected polypeptide may not be absolutely efficient. Hence, siRNA polynucleotides, may include one or more siRNA polynucleotide molecules that may differ (e.g., by nucleotide insertion or deletion) in length by one, two, three, four or more base pairs as a consequence of the variability in processing, in biosynthesis, or in artificial synthesis of the siRNA. The siRNA polynucleotide of the present invention may also comprise a polynucleotide sequence that exhibits variability by differing (e.g., by nucleotide substitution, including transition or transversion) at one, two, three or four nucleotides from a particular sequence. These differences can occur at any of the nucleotide positions of a particular siRNA polynucleotide sequence, depending on the length of the molecule, whether situated in a sense or in an antisense strand of the double-stranded polynucleotide. The nucleotide difference may be found on one strand of a double-stranded polynucleotide, where the complementary nucleotide with which the substitute nucleotide would typically form hydrogen bond base pairing, may not necessarily be correspondingly substituted. In preferred embodiments, the siRNA polynucleotides are homogeneous with respect to a specific nucleotide sequence.

One skilled in the art will readily appreciate that as a result of the degeneracy of the genetic code, many different nucleotide sequences may encode the same polypeptide. That is, an amino acid may be encoded by one of several different codons, and a person skilled in the art can readily determine that while one particular nucleotide sequence may differ from another, the polynucleotides may in fact encode polypeptides with identical amino acid sequences. As such, polynucleotides that vary due to differences in codon usage are specifically contemplated by the present invention.

One skilled in the art will appreciate, based on the disclosure provided herein, that one way to increase the mRNA and/or protein levels of SALM5, HVEM and functional equivalents thereof in a cell is by reducing or inhibiting the regulators that inhibit the expression of SALM5, HVEM and functional equivalents thereof thereby increasing the expression of SALM5, HVEM and functional equivalents thereof. Thus, the protein level SALM5, HVEM and functional equivalents thereof in a cell can also be increased.

In a preferred embodiment, the modulating sequence is an antisense nucleic acid sequence which is expressed by a plasmid vector. The antisense expressing vector is used to transfect a mammalian cell or the mammal itself, thereby causing reduced endogenous expression of a desired regulator of SALM5, HVEM and functional equivalents thereof in the cell. However, the invention should not be construed to be limited to inhibiting expression of a regulator of SALM5, HVEM and functional equivalents thereof by transfection of cells with antisense molecules. Rather, the invention encompasses other methods known in the art for inhibiting expression or activity of a protein in the cell including, but not limited to, the use of a ribozyme, the expression of a non-functional regulator (i.e. transdominant negative mutant) and use of an intracellular antibody.

Antisense molecules and their use for inhibiting gene expression are well known in the art (see, e.g., Cohen, 1989, In: Oligodeoxyribonucleotides, Antisense Inhibitors of Gene Expression, CRC Press). Antisense nucleic acids are DNA or RNA molecules that are complementary, as that term is defined elsewhere herein, to at least a portion of a specific mRNA molecule (Weintraub, 1990, Scientific American 262: 40). In the cell, antisense nucleic acids hybridize to the corresponding mRNA, forming a double-stranded molecule thereby inhibiting the translation of genes.

The use of antisense methods to inhibit the translation of genes is known in the art, and is described, for example, in Marcus-Sakura (1988, Anal. Biochem. 172: 289). Such antisense molecules may be provided to the cell via genetic expression using DNA encoding the antisense molecule as taught by Inoue, 1993, U.S. Pat. No. 5,190,931.

Alternatively, antisense molecules of the invention may be made synthetically and then provided to the cell. Antisense oligomers of between about 10 to about 30, and more preferably about 15 nucleotides, are preferred, since they are easily synthesized and introduced into a target cell. Synthetic antisense molecules contemplated by the invention include oligonucleotide derivatives known in the art which have improved biological activity compared to unmodified oligonucleotides (see U.S. Pat. No. 5,023,243).

Ribozymes and their use for inhibiting gene expression are also well known in the art (see, e.g., Cech et al., 1992, J. Biol. Chem. 267: 17479-17482; Hampel et al., 1989, Biochemistry 28: 4929-4933; Eckstein et al., International Publication No. WO 92/07065; Altman et al., U.S. Pat. No. 5,168,053). Ribozymes are RNA molecules possessing the ability to specifically cleave other single-stranded RNA in a manner analogous to DNA restriction endonucleases. Through the modification of nucleotide sequences encoding these RNAs, molecules can be engineered to recognize specific nucleotide sequences in an RNA molecule and cleave it (Cech, 1988, J. Amer. Med. Assn. 260: 3030). A major advantage of this approach is the fact that ribozymes are sequence-specific. There are two basic types of ribozymes, namely, tetrahymena-type (Hasselhoff, 1988, Nature 334: 585) and hammerhead-type. Tetrahymena-type ribozymes recognize sequences which are four bases in length, while hammerhead-type ribozymes recognize base sequences 11-18 bases in length. The longer the sequence, the greater the likelihood that the sequence will occur exclusively in the target mRNA species. Consequently, hammerhead-type ribozymes are preferable to tetrahymena-type ribozymes for inactivating specific mRNA species, and 18-base recognition sequences are preferable to shorter recognition sequences which may occur randomly within various unrelated mRNA molecules.

Ribozymes useful for inhibiting the expression of a regulator of SALM5, HVEM and functional equivalents thereof may be designed by incorporating target sequences into the basic ribozyme structure which are complementary to the mRNA sequence of the desired regulator of SALM5, HVEM and functional equivalents. Ribozymes targeting the desired regulator of SALM5, HVEM and functional equivalents may be synthesized using commercially available reagents (Applied Biosystems, Inc., Foster City, Calif.) or they may be genetically expressed from DNA encoding them.

### Methods

SALM5-HVEM antagonists, nucleic acids encoding SALM5-HVEM antagonists, or cells expressing SALM5-HVEM antagonists can be used to increase or promote inflammation in the CNS or other immune privileged tissue. Such an increase in inflammation is useful in the treatment of, for example, neoplasm (e.g., cancer, tumor, etc.) or infection (e.g., bacterial, viral, fungal, protozoan, etc.), in an immune privileged tissue.

SALM5-HVEM agonists, nucleic acids encoding SALM5-HVEM agonists, or cells expressing SALM5-HVEM agonists can be used to reduce or inhibit inflammation in the CNS or other immune privileged tissue, including, but not limited to, inflammation due to mechanical trauma (e.g. traumatic brain injury), ischemia (e.g. stroke) and infection (e.g. bacterial, viral, fungal, protozoan, etc.), autoimmunity, and inflammatory disease.

Whether the invention is directed to agonists or antagonists, the methods can include contacting a cell with a composition of the invention. The contacting can be in vitro, ex vivo, or in vivo (e.g., in a mammal such as a mouse, rat, rabbit, dog, cow, pig, non-human primate, or a human).

The SALM5-HVEM antagonists provided herein are generally useful as immune response-inducing therapeutics. In general, the compositions are useful for treating a subject having or being predisposed to any disease or disorder to which the subject's immune system fails or is required to mount an immune response. The ability of SALM5-HVEM antagonists to induce an immune response (e.g., inflammation) makes the disclosed compositions useful to increase or promote immune responses involving at least a myeloid cell.

Therefore, where it is desirable to increase the immune response in the CNS or other immune privileged tissue, the antagonists of the invention can be administered to a host in need thereof an effective amount of one or more SALM5-HVEM antagonists. In one embodiment, reducing or inhibiting SALM5-HVEM binding and associated biological activities is accomplished by providing one or more SALM5-HVEM antagonist that decreases or inhibits binding of SALM5 to HVEM. In another embodiment, SALM5 and/or HVEM expression is downregulated by providing one or more inhibitory nucleic acids including, but not limited to, ribozymes, triplex-forming oligonucleotides (TFOs), antisense DNA, siRNA, and microRNA specific for nucleic acids encoding SALM5 or HVEM. SALM5-HVEM antagonists can also be provided in combination with other immunomodulatory agents.

The SALM5-HVEM agonists provided herein are generally useful as immune response-reducing therapeutics. In general, the compositions are useful for treating a subject having or being predisposed to any disease or disorder to which the subject's immune system mounts an immune response. The ability of SALM5-HVEM agonists to reduce an immune response (e.g., inflammation) makes the disclosed compositions useful to reduce or inhibit immune responses involving at least a myeloid cell. The agonists of the invention can be used to alleviating, preventing and/or eliminating one or more symptoms associated with inflammatory immune responses, autoimmune disorders or immune responses to grafts, including graft-versus-host disease.

Therefore, one embodiment of a method for treating or inhibiting immunologic disorders, including inflammatory disorders, autoimmune disorders, and immune responses involved in graft rejection, including graft-versus-host disease, is by promoting or increasing the binding of SALM5 to HVEM and by inducing their activity. For example, the method can be by administering to a host in need thereof an effective amount of one or more SALM5-HVEM agonists.

In some in vivo approaches, the compositions of the invention can be administered to a subject in a therapeutically effective amount. Typically, the polypeptides can be suspended in a pharmaceutically-acceptable carrier. Pharmaceutically acceptable carriers are biologically compatible vehicles (e.g., physiological saline) that are suitable for administration to a human. A therapeutically effective amount is an amount of a composition that is capable of producing a medically desirable result in a treated animal. Compositions of the invention can be administered orally or by intravenous infusion, or injected subcutaneously, intramuscularly, intraperitoneally, intrarectally, intravaginally, intranasally, intragastrically, intratracheally, or intrapulmonarily. The compositions of the invention can be delivered directly to an appropriate lymphoid tissue (e.g., spleen, lymph node, or mucosal-associated lymphoid tissue) or directly to an organ or tissue graft.

Nucleic acids molecules of the invention can be administered to subjects in need thereof. Nucleic delivery involves introduction of “foreign” nucleic acids into a cell and ultimately, into a live animal. Several general strategies for gene therapy have been studied and have been reviewed extensively (Yang, N-S., Crit. Rev. Biotechnol. 12: 335-356 (1992); Anderson, W. F., Science 256: 808-813 (1992); Miller, A. S., Nature 357: 455-460 (1992); Crystal, R. G., Amer. J. Med. 92 (suppl 6A): 44S-52S (1992); Zwiebel, J. A. et al., Ann. N.Y. Acad. Sci. 618: 394-404 (1991); McLachlin, J. R. et al., Prog. Nuc. Acid Res. Molec. Biol. 38: 91-135 (1990); Kohn, D. B. et al., Cancer Invest. 7: 179-192 (1989), which references are herein incorporated by reference in their entirety).

One approach includes nucleic acid transfer into primary cells in culture followed by autologous transplantation of the ex vivo transformed cells into the host, either systemically or into a particular organ or tissue. In one embodiment, vectors containing nucleic acids of the invention are transfected into cells that are administered to a subject in need thereof.

Ex vivo methods can include, for example, the steps of harvesting cells from a subject, culturing the cells, transducing them with an expression vector, and maintaining the cells under conditions suitable for expression of the polypeptides provided herein. These methods are known in the art of molecular biology. The transduction step can be accomplished by any standard means used for ex vivo gene therapy, including, for example, calcium phosphate, lipofection, electroporation, viral infection, and biolistic gene transfer. Alternatively, liposomes or polymeric microparticles can be used. Cells that have been successfully transduced then can be selected, for example, for expression of the coding sequence or of a drug resistance gene. The cells then can be lethally irradiated (if desired) and injected or implanted into the subject.

Nucleic acid therapy can be accomplished by direct transfer of a functionally active DNA into mammalian somatic tissue or organ in vivo. For example, nucleic acids of the invention can be administered directly to lymphoid tissues. Alternatively, lymphoid tissue specific targeting can be achieved using lymphoid tissue-specific transcriptional regulatory elements (TREs) such as a B lymphocyte-, T lymphocyte-, or dendritic cell-specific TRE. Lymphoid tissue specific TREs include, for example, those known in the art [see, e.g., Thompson et al. (1992) Mol. Cell. Biol. 12: 1043-1053; Todd et al. (1993) J. Exp. Med. 177: 1663-1674; and Penix et al. (1993) J. Exp. Med. 178: 1483-1496].

### Pharmaceutical

The therapeutic and prophylactic methods of the invention thus encompass the use of pharmaceutical compositions. The pharmaceutical compositions useful for practicing the invention may be administered to deliver a dose of between 1 ng/kg/day and 100 mg/kg/day. In one embodiment, the invention envisions administration of a dose which results in a concentration of the compound of the present invention between 1 μM and 10 μM in a mammal.

Typically, dosages which may be administered in a method of the invention to an animal, preferably a human, range in amount from 0.5 μg to about 50 mg per kilogram of body weight of the animal. While the precise dosage administered will vary depending upon any number of factors, including but not limited to, the type of animal and type of disease state being treated, the age of the animal and the route of administration. Preferably, the dosage of the compound will vary from about 1 μg to about 10 mg per kilogram of body weight of the animal. More preferably, the dosage will vary from about 3 μg to about 1 mg per kilogram of body weight of the animal.

The compound may be administered to an animal as frequently as several times daily, or it may be administered less frequently, such as once a day, once a week, once every two weeks, once a month, or even less frequently, such as once every several months or even once a year or less. The frequency of the dose will be readily apparent to the skilled artisan and will depend upon any number of factors, such as, but not limited to, the type and severity of the disease being treated, the type and age of the animal, etc. The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

Although the description of pharmaceutical compositions provided herein are principally directed to pharmaceutical compositions which are suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation. Subjects to which administration of the pharmaceutical compositions of the invention is contemplated include, but are not limited to, humans and other primates, mammals including commercially relevant mammals such as non-human primates, cattle, pigs, horses, sheep, cats, and dogs.

Pharmaceutical compositions that are useful in the methods of the invention may be prepared, packaged, or sold in formulations suitable for ophthalmic, oral, rectal, vaginal, parenteral, topical, pulmonary, intranasal, buccal, or another route of administration. Other contemplated formulations include projected nanoparticles, liposomal preparations, resealed erythrocytes containing the active ingredient, and immunologically-based formulations.

A pharmaceutical composition of the invention may be prepared, packaged, or sold in bulk, as a single unit dose, or as a plurality of single unit doses. As used herein, a “unit dose” is discrete amount of the pharmaceutical composition comprising a predetermined amount of the active ingredient. The amount of the active ingredient is generally equal to the dosage of the active ingredient which would be administered to a subject or a convenient fraction of such a dosage such as, for example, one-half or one-third of such a dosage.

The relative amounts of the active ingredient, the pharmaceutically acceptable carrier, and any additional ingredients in a pharmaceutical composition of the invention will vary, depending upon the identity, size, and condition of the subject treated and further depending upon the route by which the composition is to be administered. By way of example, the composition may comprise between 0.1% and 100% (w/w) active ingredient.

In addition to the active ingredient, a pharmaceutical composition of the invention may further comprise one or more additional pharmaceutically active agents. Other active agents useful in the treatment of fibrosis include anti-inflammatories, including corticosteroids, and immunosuppressants.

Controlled- or sustained-release formulations of a pharmaceutical composition of the invention may be made using conventional technology.

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, intraocular, intravitreal, subcutaneous, intraperitoneal, intramuscular, intrasternal injection, intratumoral, and kidney dialytic infusion techniques.

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multi-dose containers containing a preservative. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In one embodiment of a formulation for parenteral administration, the active ingredient is provided in dry (i.e. powder or granular) form for reconstitution with a suitable vehicle (e.g. sterile pyrogen-free water) prior to parenteral administration of the reconstituted composition.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally-acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides. Other parentally-administrable formulations which are useful include those which comprise the active ingredient in microcrystalline form, in a liposomal preparation, or as a component of a biodegradable polymer system. Compositions for sustained release or implantation may comprise pharmaceutically acceptable polymeric or hydrophobic materials such as an emulsion, an ion exchange resin, a sparingly soluble polymer, or a sparingly soluble salt.

A pharmaceutical composition of the invention may be prepared, packaged, or sold in a formulation suitable for pulmonary administration via the buccal cavity. Such a formulation may comprise dry particles which comprise the active ingredient and which have a diameter in the range from about 0.5 to about 7 nanometers, and preferably from about 1 to about 6 nanometers. Such compositions are conveniently in the form of dry powders for administration using a device comprising a dry powder reservoir to which a stream of propellant may be directed to disperse the powder or using a self-propelling solvent/powder-dispensing container such as a device comprising the active ingredient dissolved or suspended in a low-boiling propellant in a sealed container. Preferably, such powders comprise particles wherein at least 98% of the particles by weight have a diameter greater than 0.5 nanometers and at least 95% of the particles by number have a diameter less than 7 nanometers. More preferably, at least 95% of the particles by weight have a diameter greater than 1 nanometer and at least 90% of the particles by number have a diameter less than 6 nanometers. Dry powder compositions preferably include a solid fine powder diluent such as sugar and are conveniently provided in a unit dose form.

Low boiling propellants generally include liquid propellants having a boiling point of below 65° F. at atmospheric pressure. Generally the propellant may constitute 50 to 99.9% (w/w) of the composition, and the active ingredient may constitute 0.1 to 20% (w/w) of the composition. The propellant may further comprise additional ingredients such as a liquid non-ionic or solid anionic surfactant or a solid diluent (preferably having a particle size of the same order as particles comprising the active ingredient).

Pharmaceutical compositions of the invention formulated for pulmonary delivery may also provide the active ingredient in the form of droplets of a solution or suspension. Such formulations may be prepared, packaged, or sold as aqueous or dilute alcoholic solutions or suspensions, optionally sterile, comprising the active ingredient, and may conveniently be administered using any nebulization or atomization device. Such formulations may further comprise one or more additional ingredients including, but not limited to, a flavoring agent such as saccharin sodium, a volatile oil, a buffering agent, a surface active agent, or a preservative such as methylhydroxybenzoate. The droplets provided by this route of administration preferably have an average diameter in the range from about 0.1 to about 200 nanometers.

The formulations described herein as being useful for pulmonary delivery are also useful for intranasal delivery of a pharmaceutical composition of the invention.

Another formulation suitable for intranasal administration is a coarse powder comprising the active ingredient and having an average particle from about 0.2 to 500 micrometers. Such a formulation is administered in the manner in which snuff is taken i.e. by rapid inhalation through the nasal passage from a container of the powder held close to the nares.

Formulations suitable for nasal administration may, for example, comprise from about as little as 0.1% (w/w) and as much as 100% (w/w) of the active ingredient, and may further comprise one or more of the additional ingredients described herein.

A pharmaceutical composition of the invention may be prepared, packaged, or sold in a formulation suitable for buccal administration. Such formulations may, for example, be in the form of tablets or lozenges made using conventional methods, and may, for example, 0.1 to 20% (w/w) active ingredient, the balance comprising an orally dissolvable or degradable composition and, optionally, one or more of the additional ingredients described herein. Alternately, formulations suitable for buccal administration may comprise a powder or an aerosolized or atomized solution or suspension comprising the active ingredient. Such powdered, aerosolized, or aerosolized formulations, when dispersed, preferably have an average particle or droplet size in the range from about 0.1 to about 200 nanometers, and may further comprise one or more of the additional ingredients described herein.

As used herein, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” which may be included in the pharmaceutical compositions of the invention are known in the art and described, for example in Remington's Pharmaceutical Sciences (1985, Genaro, ed., Mack Publishing Co., Easton, Pa.), which is incorporated herein by reference.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the compounds of the present invention and practice the claimed methods. The following working examples therefore, specifically point out the preferred embodiments of the present invention, and are not to be construed as limiting in any way the remainder of the disclosure.

### Example 1

### Interaction of SALM5 and HVEM Limits Inflammation in the Central Nervous System

The central nervous system (CNS) is an immune-privileged organ with capacity to prevent overactive inflammation. In addition to blood brain barrier, active immune suppressive mechanisms operate and underlying molecular mechanism remains largely unclear. The results presented herein discuss the role of a key suppressive molecule in the CNS: synaptic adhesion-like molecule 5 (SALM5), an adhesion molecule exclusively expressed by neuronal cells. Administration of SALM5 monoclonal antibody (mAb) aggravated clinical symptoms in mouse experimental autoimmune encephalomyelitis (EAE), accompanied with massive infiltration of macrophages and CD4+ T cells in the CNS. The immunomodulatory effect of SALM5 is restricted to CNS and caused by transmitting a suppressive signal to microglia and macrophages, which in turn inhibits the expansion of pathogenic T cells.

The results presented here demonstrate that Herpes virus entry mediator (HVEM) in blood borne cells was identified as the counter-receptor for SALM5. It was also demonstrated that the interaction of SALM5 and HVEM is essential for suppressing CNS inflammatory immune responses. The findings presented herein provide a unique mechanism refraining immune response in the CNS and provide new therapeutic targets for the control of CNS diseases.

Briefly, a monoclonal antibody (mAb) specific for SALM5 was generated and subsequently showed that administration of this SALM5 mAb exacerbated EAE, leading to profound leukocyte infiltration and enhanced inflammation in the CNS. Consistent with the CNS-restricted expression pattern of SALM5, T cell responses to antigens were not altered in peripheral lymphoid organs. With extensive screening of over 2,300 individually displayed cell surface proteins in a Receptor Array system (Yao et al., 2011, Immunity 34: 729-740), HVEM, (also known as TNFRSF14) a member of tumor necrosis factor receptor (TNFR) superfamily, was identified as the endogenous counter-receptor for SALM5. HVEM (Montgomery et al., 1996, Cell 87: 427-436) is ubiquitously expressed on all lymphoid and myeloid cells and it differentially modulates T cell response by engaging different binding partners (Murphy et al., 2010, Annu. Rev. Immunol. 28: 389-411). By interacting with ligand-SALM5, a TNF-like molecule, HVEM delivers a costimulatory signal for T cell activation (Tamada et al., 2000, Nat. Med. 6: 283-289). On the other hand, HVEM may bind both BTLA and CD160 receptors, two immunoglobulin (Ig) superfamily molecules, to inhibit T cell responses (Cai et al., 2008, Nat. Immunol. 9: 176-185; Sedy et al., 2005, Nat. Immunol. 6: 90-98).

Using HVEM-deficient mice, it was demonstrated that HVEM is the counter-receptor responsible for the effect of SALM5 mAb on EAE. Although not wishing to be bound by any particular theory, it is believed that the SALM5-HVEM interaction contributes to the immune suppressive aspects of CNS.

The materials and methods employed in these experiments are now described.

### Materials and Methods

Animals, antibodies and recombinant proteins

Female C57BL/6 mice in 6-10 weeks of age were purchased from National Cancer Institute, NIH (Frederick, Md.); 2D2 TCR-transgenic mice were purchased from the Jackson Laboratory (Bar Harbor, Me.). HVEM−/− mice in C57BL/6 background have been described (Tao et al., 2008, J. Immunol. 180: 6649-6655). All fusion proteins were generated by fusing extracellular domain of each molecule with mouse or human FCC tag (Dong et al., 1999, Nat. Med. 5: 1365-1369). Hamster mAbs against mouse SALM5 were generated by immunizing a hamster with mouse SALM5-Ig fusion protein. All antibodies for flow cytometry staining, if not specified, were purchased from BD Bioscience (San Jose, Calif.) or eBioscience (San Diego, Calif.).

CDS Screening

The detailed method for high-throughput screening by cellular detection system (CDS) has been reported previously (Yao et al., 2011, Immunity 34: 729-740). Briefly, plasmids containing over 2300 human transmembrane genes were diluted by OPTI-MEM media and placed individually into five 384-well plates at 60 ng/well.

Lipofectamine 2000 was added to each well and mixed with plasmids for 30 minutes. Ten thousand HEK293T cells were added subsequently to each well to perform transient transfection. Eight hours after transfection, 50 ng human SALM5-Ig or HVEM-Ig and 50 ng anti-human Ig or anti-mouse Ig FMAT blue secondary antibody were added into each well. The plates were read twenty-four hours after transfection by the Applied Biosystems 8200 cellular detection system and analyzed by CDS 8200 software. Each plate has a well containing human Fc Receptor as an internal positive control for transfection.

Plasmids for mouse HVEM and SALM5 mutants

Mouse ΔHVEM was made by PCR method similar to what was described before (Sedy et al., 2005, Nat. Immunol. 6: 90-98). Point mutations on HVEM were selected according to previous publications (Cheung et al., 2005, Proc. Natl. Acad. Sci. USA 102: 13218-13223; Compaan et al., 2005, Nat. Med. 5: 1365-1369). HVEM and SALM5 mutants were generated by PCR method.

EAE Model

C57BL/6 mice of 8-12 weeks old were immunized subcutaneously on day 0 with 100 μg MOG peptide (35-55) emulsified in complete Freund's adjuvant (CFA) (Difco, Lawrence, Kans.). 400 ng Pertussis toxin (Sigma Aldrich, St. Louis, Mo.) in 200 μl PBS was injected twice, on days 0 and 2. Each mouse was injected intraperitoneally with 300 μg SALM5 mAb or control antibody in 300 μl PBS on days 1 and 4. In some experiments, antibody was injected on days 10, 14 and 17. Disease severity was scored on the following scale: 0, no disease; 1, tail paralysis; 2, paraparesis; 3, paraplegia; 4, paraplegia with forelimb weakness or paralysis; 5, moribund or dead, as described (Stromnes et al., 2006, Nat. Protoc. 1: 1810-1819). In some experiments, ˜2×10⁶ naïve Thy1.1+2D2-transgenic T cells were transferred intravenously into mice 24 hours before immunization. BrdU were intraperitoneally injected into mice 24 hours before analysis.

LPS Administration

B6 mice treated with pertossis toxin on days 0 and 2 were injected intraperitoneally with SALM5 mAb or control antibody on days 1 and 4. On day 5, each mouse received a single intravenous dose of LPS (0.5 mg/kg) 24 hours before sacrificed for Iba-1 staining.

Isolation of CNS Mononuclear Cells

Sacrificed mice were perfused with cold PBS before brains and spine cords were dissected. The tissues were homogenized and digested with collagenase D and DNase I in 37° C. for 45 minutes. After centrifugation, pellets were resuspended in 30% Percoll and carefully layered on the top of 70% Percoll. The Percoll gradient was centrifuged in room temperature at 1,000 g for 30 minutes without brake. Mononuclear cells at the 30% and 70% Percoll interphase layer were harvested and washed with complete RPMI 1640 before FACS staining or in vitro culture. Microglia/macrophages were isolated by removing non-adherent cells after two hour culture in 37 degree.

Histology and Immunohistochemistry

Tissues were removed from naïve mice or mice with EAE and embedded in paraffin. Tissues were cut into sections 5 μm in thickness and stained with H&E method to reveal inflammatory infiltrates. For immunohistochemistry, deparaffinized sections were stained with anti-CD3 (AbD Serotic, Clone CD3-12), anti-MAG3 (AbD Serotic, Clone M3/84), and anti-Iba1 (Wako Pure Chemical Industries, Japan) according to manufacturer's protocols. For SALM5 staining, tissues were deparaffinized and rehydrated prior to antigen retrieval in citrate buffer. Tissues were then stained with different SALM5 antibodies, followed by incubation with amplification system k1500 (DakoCytomation, Glostrup, Denmark). After HRP staining, slides were visualized with 3-3′ diaminobenzidine (Sigma Aldrich, St. Louis, Mo.).

T Cell Proliferation and Cytokine Assays

For ex vivo re-stimulation by MOG 35-55 peptide, draining lymph nodes were collected from immunized mice and single-cell suspensions were prepared by mechanical disruption. 3×10⁶/ml lymphocytes were cultured in RPMI 1640 medium with or without MOG peptide for 72 hours. Proliferation was measured by [H³] thymidine incorporation (Amersham Biosciences, Piscataway, N.J.) at the last 12 hours of cell culture. Supernatant after 72 hours culture was measured for cytokine production by mouse TH1/TH2/TH17 cytokine CBA kit (BD Biosciences, San Jose, Calif.).

Statistical Analysis

Student's t-test was used for statistical analysis, and P values reflect comparison with the control sample. P values less than 0.05 were considered statistically significant. The error bars in figures represent Standard Deviation (SD).

The results of the experiments are now described.

### Blockade of SALM5 by mAb Aggravates Disease in a Mouse EAE Model

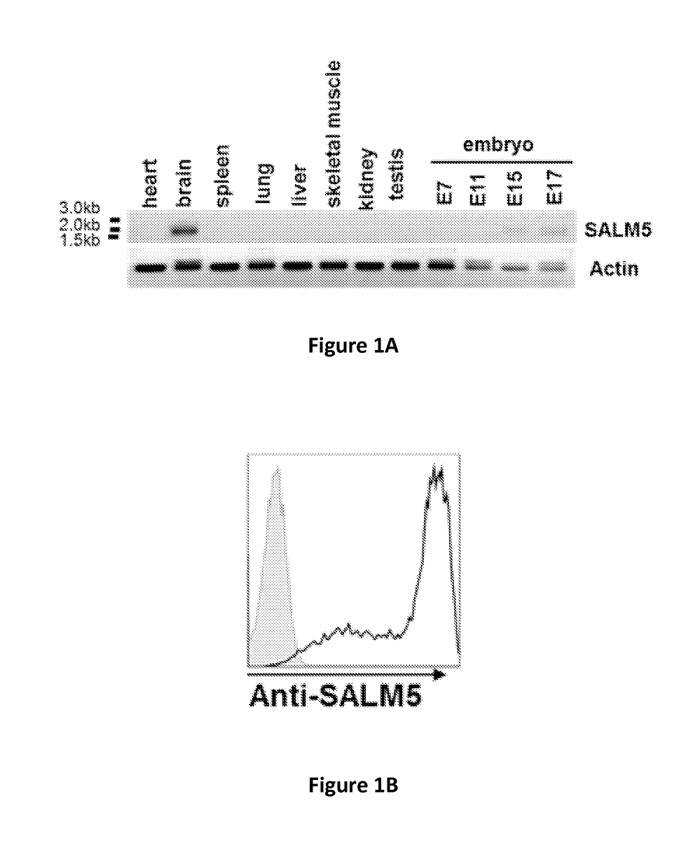

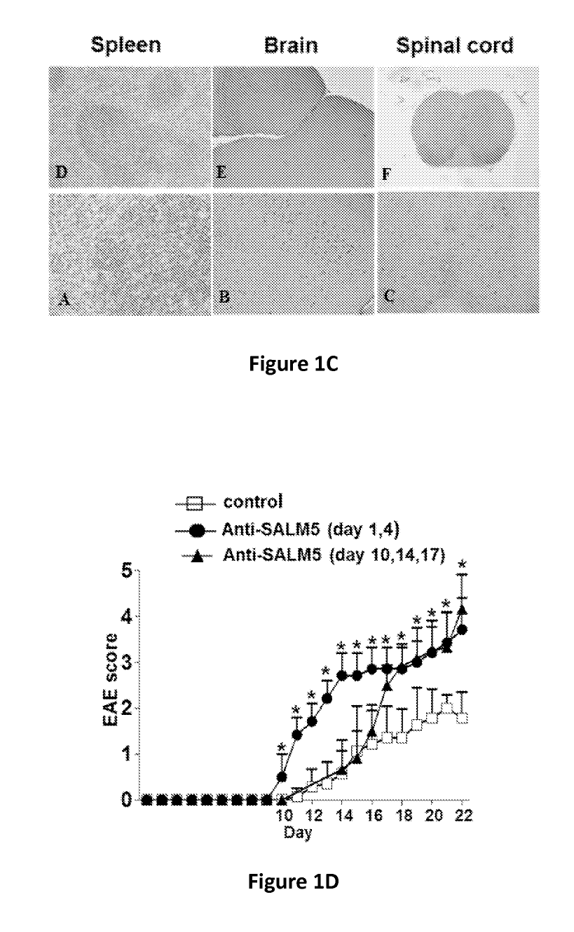

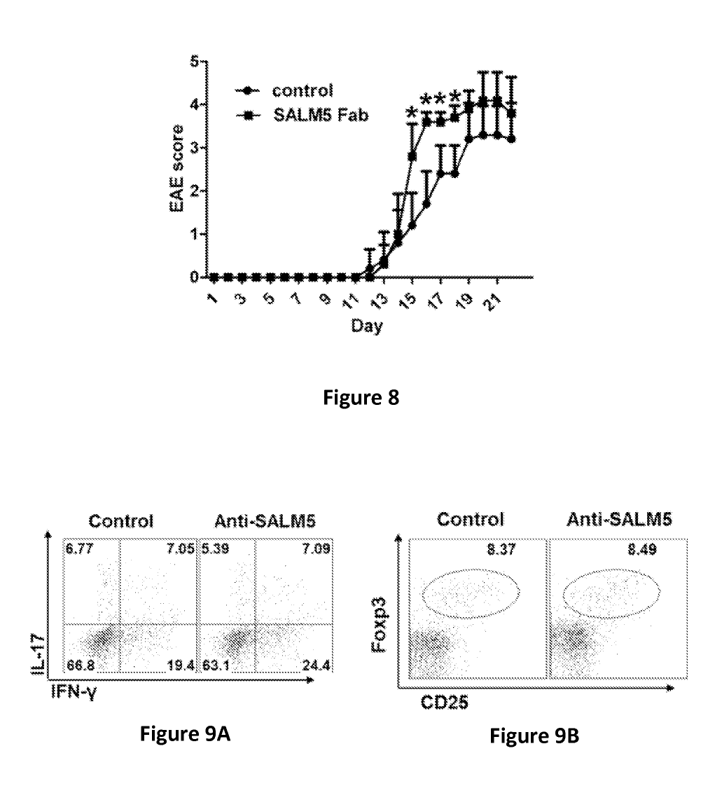

The CNS-restricted expression pattern of SALM5 suggests an organ-specific function. By a highly sensitive RT-PCR method, it was demonstrated that SALM5 mRNA is detected in the brain, but not other organs including heart, spleen, lung, liver, and skeleton muscle (FIG. 1A). To explore the function of SALM5, a monoclonal antibody (mAb) against mouse SALM5 was generated by immunizing a hamster with recombinant SALM5 protein. The specificity of the mAb, produced by a hybridoma clone 7A10 was confirmed by flow cytometry: 7A10 only bound to HEK293T cells transfected with mouse SALM5, but not mock-transfected control cells (FIG. 1B). The specificity of this mAb was further verified by ELISA. Immunostaining of SALM5 indicated that SALM5 is constitutively expressed in the CNS, but not spleen (FIG. 1C), and the staining pattern is consistent with two commercial SALM5 antibodies (FIG. 7). Subsequently, experiments were performed to test the effect of SALM5 mAb in a mouse EAE model of human multiple sclerosis. In this model, peripheral leucocytes infiltrate the CNS to cause inflammation and neuron damage (Kuchroo et al., 2002, Ann. Rev. Immunol. 20: 101-123). EAE was induced by immunizing mice with myelin oligodendrocyte glycoprotein peptide (MOG) (35-55) emulsified in complete Freund's adjuvant (CFA). In this study, mice were treated with either purified 7A10 mAb or hamster IgG as the control after immunization. MOG-immunized mice developed symptoms of EAE approximately 10 days after induction, which manifested as progressive paralysis of their limbs Stromnes and Goverman, 2006, Nat. Protoc. 1: 1810-1819) Though disease incidence rates in both groups were similar, mice treated with SALM5 mAb during priming (day 1 and day 4) developed disease symptoms earlier than their counterparts injected with control mAb. In addition, SALM5 mAb-treated mice had significantly worse clinical appearances (FIG. 1D). Half of the mice treated with SALM5 mAb died, whereas all of the mice treated with the control antibody survived. Importantly, delayed treatment of SALM5 mAb 10 days after MOG immunization could still aggravate EAE symptoms and the severity of EAE disease reached similar level as the mice treated with SALM5 mAb at priming phase. To further exclude the possibility of agonistic effect of SALM5 mAb, (F(ab)'2 fragment was generated from SALM5 mAb, and tested its effect on EAE disease. Similar to the parental antibody, treatment of SALM5 F(ab)'2 fragment significantly aggravated EAE diseases, further excluding its in vivo effect as agonist for SALM5 (FIG. 8). Altogether, although not wishing to be bound by any particular theory, these observations support a suppressive function of SALM5 in the development of EAE diseases in both priming and effector phases.

### Blockade of SALM5 Leads to Massive Increase of CD4+ T Cells and Macrophages in the CNS

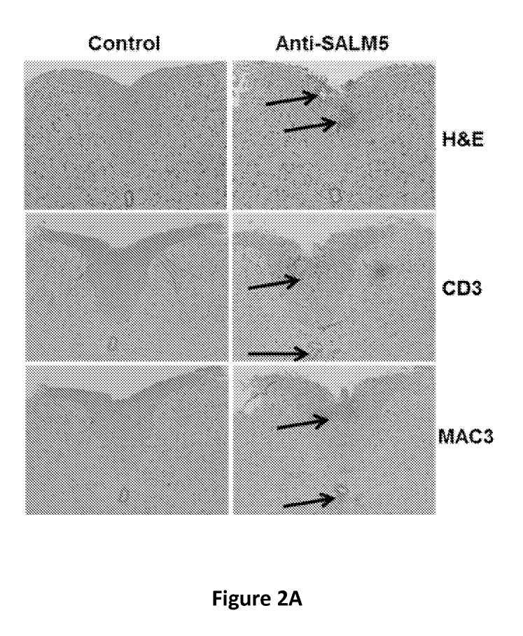

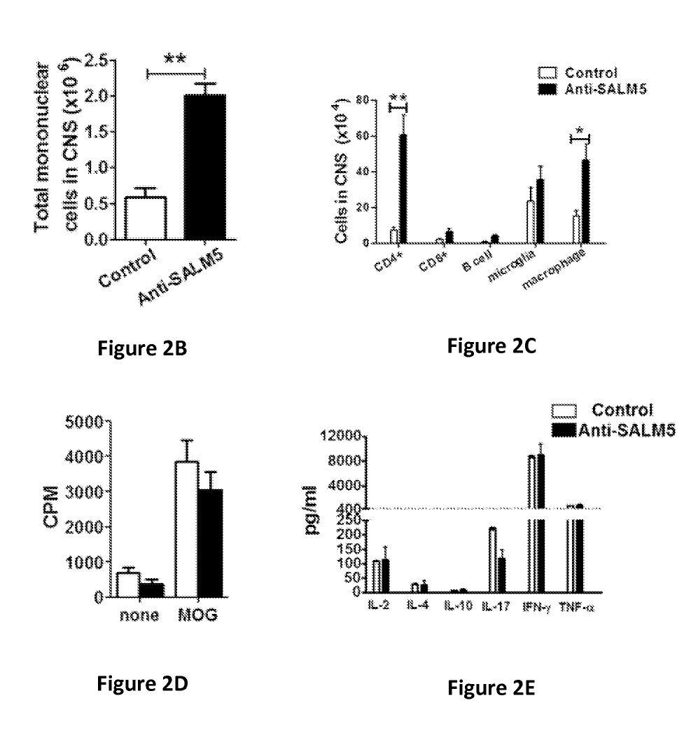

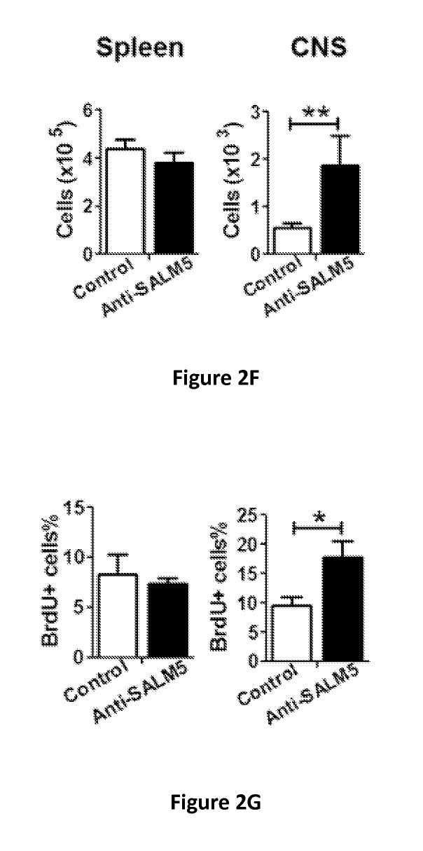

Staining of spinal cord sections with H&E in the mice treated with SALM5 mAb demonstrated extensive inflammation, characterized by massive infiltration of mononuclear cells (as indicated by arrows) whereas mice treated with control antibody did not. Immunochemical staining demonstrated that the inflammatory cells in spinal cord were predominantly CD3+ T cells and Mac3+ macrophages (FIG. 2A). Flow cytometry analyses of isolated mononuclear cells from EAE mouse CNS tissue indicate approximately four times as many infiltrating CD45+ cells in mice treated with SALM5 mAb compared with control mice (FIG. 2B). Of those CD45+ cells, the increase of CD4+ T cells and CD11b+CD45^{hi} macrophages accounts for the majority of total cell number increase in SALM5 mAb-treated mice, a result consistent with immunochemical staining (FIG. 2A). The numbers of infiltrating CD8+ T cells, B220+ B cells and CD11b+CD45^{int} microglia remained similar in two groups (FIG. 2C).

### Selective Expansion of MOG-Specific T Cells in the CNS but not in Peripheral Lymphoid Organs by SALM5 Blockade

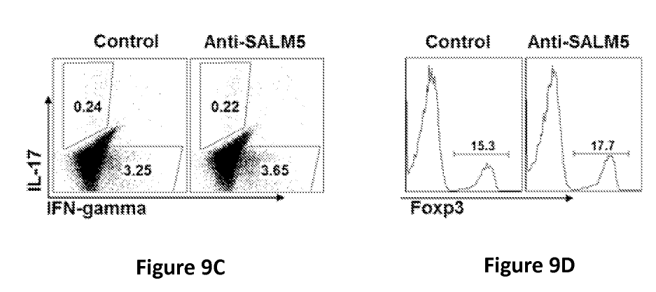

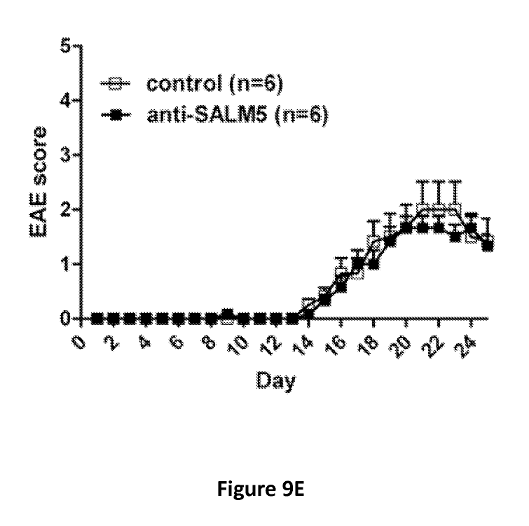

Ample evidence supports that MOG-specific CD4+ T cells, especially Th1 (IFN-γ producing) and Th17 (IL-17 producing) subsets, play central role in the pathogenesis of EAE, whereas regulatory T cells (CD4+ Foxp3+ Treg) negatively regulate EAE (Kohm et al., 2002, J. Immunol. 169: 4712-4716). The ratios of these T cells in the CNS were first examined after SALM5 mAb treatment. Surprisingly, the percentages of IFN-γ or IL-17-producing cells remained similar between SALM5 mAb and control mAb treatment groups. In addition, the ratio of CD25+ Foxp3+ regulatory T cell (Treg) in the CNS also did not change (FIGS. 9A-9B). Therefore, SALM5 mAb does not seem to affect the differentiation of CD4+ T cells into functional subsets in the CNS.

Although not wishing to be bound by any particular theory, a possible interpretation for the increased T cells in the CNS after SALM5 mAb treatment is enhanced expansion of MOG-specific T cells in peripheral lymphoid organs, leading to their accumulation in the CNS. To analyze the status of MOG-specific T cells activation in peripheral lymphoid organs, draining lymph node cells were isolated nine days after MOG peptide immunization and re-stimulated with MOG (35-55) peptide in vitro. There was no significant alteration in T cell proliferation or cytokine production including IL-2, IL-4, IL-10, IL-17, IFN-γ and TNF-α upon SALM5 mAb treatment (FIGS. 2D-2E). Similar results were also obtained using splenic T cells. In addition, the percentages of Foxp3+ Treg cells, IL-17 or IFN-γ-producing T cells were all similar between mice with or without SALM5 mAb treatment, as determined by flow cytometry analysis (FIGS. 9C-9D). Splenocytes isolated from the mock- and SALM5 mAb-treated mice induced similar severity of EAE diseases upon transferred into B6-RAG knockout mice (FIG. 9E). All these results indicate that SALM5 mAb treatment has no effect on the activation and differentiation of MOG-specific T cells in lymphoid organs.

Next, a more sensitive method was employed to trace MOG-specific T cell response in peripheral lymphoid organs and CNS by transfer of naive 2D2 TCR transgenic T cells. 2D2 T cells are clonal CD4+ transgenic T cells bearing a T cell receptor specific for MOG (33-55) peptide (Bettelli et al., 2003, J. Exp. Med. 197: 1073-1081). In this system, C57BL/6 mice (Thy1.2+) were transferred with purified 2D2 cells from congeneic Thy1.1+ background and were immunized with MOG peptide to induce EAE. The mice were fed with BrdU to monitor the proliferation of 2D2 cells by gating on Thy1.1+ cells. There were significantly more infiltrating 2D2 T cells in the CNS upon SALM5 mAb treatment (FIG. 2F). Interestingly, the percentage of dividing 2D2 T cells in the CNS, measured by BrdU incorporation, was greatly increased in mice treated with SALM5 mAb (FIG. 2G), indicating that SALM5 mAb induces proliferation of MOG-specific T cells in the CNS. In sharp contrast, no difference was observed in the spleens of mice treated with either control or SALM5 mAb, in terms of the percentage of the BrdU-incorporated or the total number of 2D2 T cells (FIGS. 2F-2G). Inoculation of SALM5 mAb also had no effect on 2D2 T cell apoptosis, as measured by annexin V staining. Taken together with exclusive SALM5 expression in the CNS (FIG. 1A), SALM5 mAb, though administered systemically, selectively promotes T cell proliferation within the CNS.

SALM5 does not Directly Suppress T Cell Activation

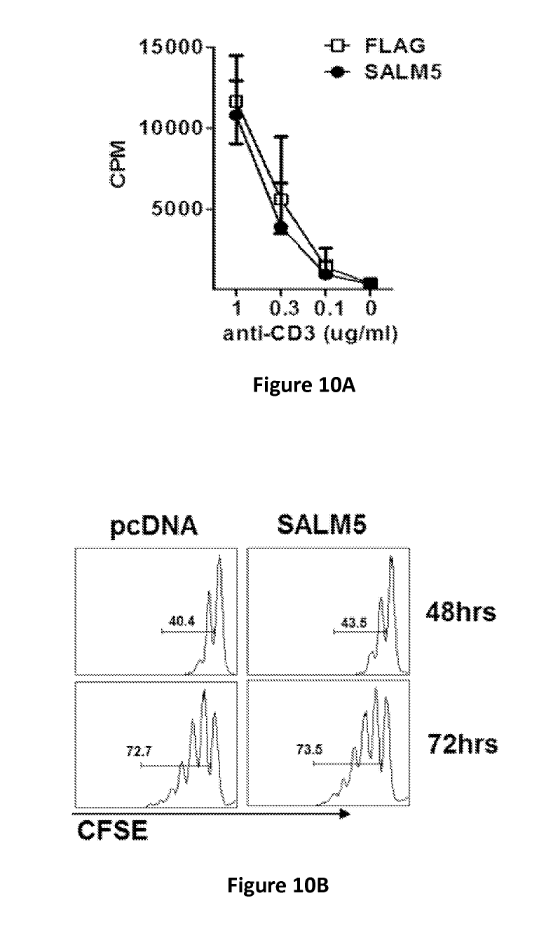

SALM5 protein was tested to determine if it could directly suppress T cell activation in vitro. Purified 2D2 transgenic T cells were stimulated with immobilized CD3 mAb as TCR mimicry in the presence of recombinant SALM5-Ig or control Ig. SALM5-Ig has no effect on 2D2 T cell proliferation as measured by H³ thymidine incorporation (FIG. 10A). A similar result was also obtained when pre-activated 2D2 T cells were used. The function of SALM5-Ig was also tested in a different setting in which HEK293T cells expressing K^{b}-OVA were transiently transfected with full-length SALM5 or control plasmid. The expression of SALM5 was confirmed by antibody staining. Transfected cells were irradiated right before incubation with CFSE-labeled OT-I T cells, a clonal TCR transgenic T cell line recognizing a chicken ovalbumin (OVA) epitope on the context of H-2K^{b} (Hogquist et al., 1994, Cell 76: 17-27). As shown in FIG. 10B, expression of SALM5 on K^{b}-OVA transfectants had no effect on OT-I T cell proliferation, as measured by dilution of CFSE. It was concluded that SALM5 does not directly suppress T cell activation. These findings also implicate that T cells are not the direct targets for SALM5 and T cell expansion in the CNS induced by SALM5 mAb may be indirectly mediated through other cells.

### SALM5 Inhibits Microglia/Macrophage-Mediated Neuroinflammation

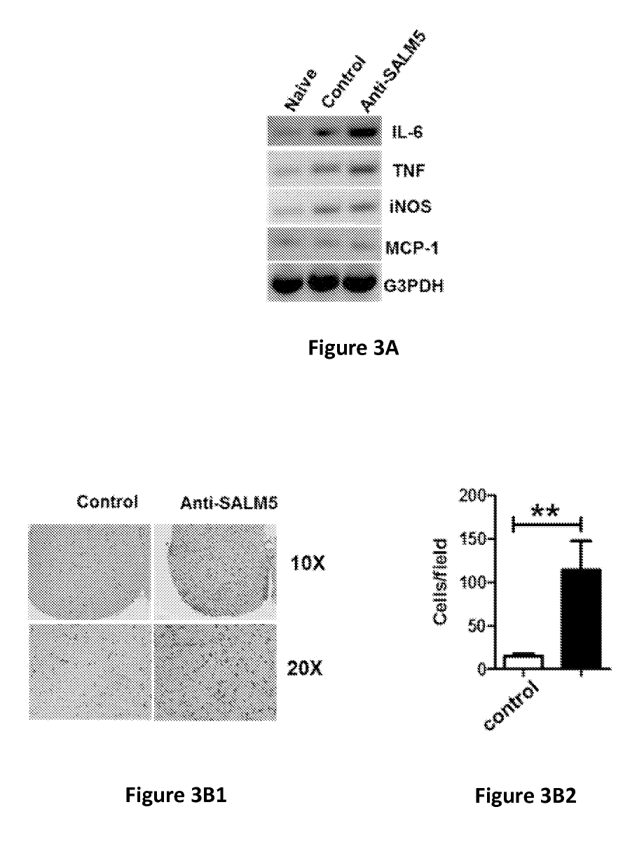

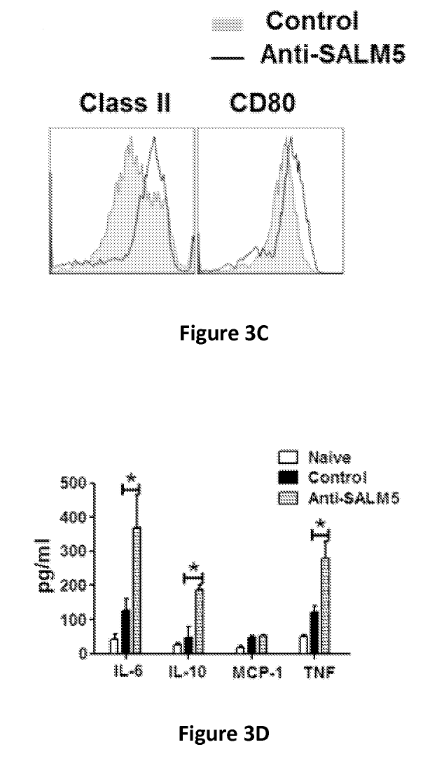

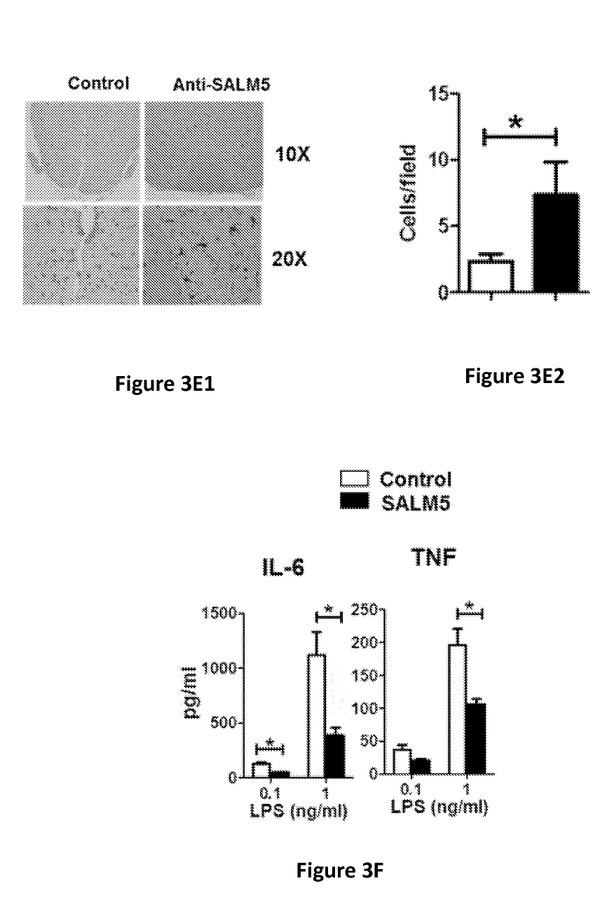

RT-PCR analysis of diseased CNS tissues revealed upregulation of proinflammatory cytokines and mediators including IL-6, TNF and iNOS after SALM5 mAb treatment (FIG. 3A). Because microglial cells and macrophages are major components of inflammatory cells in the CNS after SALM5 mAb treatment (FIGS. 2A and 2C), it was next determined whether SALM5 mAb induces activation of microglia/macrophages in the CNS by immune-staining with specific mAb against Iba1. Iba1, an ionized calcium binding adaptor protein, is a specific marker for activated macrophages and/or microglia (Heppner et al., 2005, Nat. Med. 11: 146-152; Sasaki et al., 2001, Biochem. Biophys. Res. Commun. 286: 292-297). Immunohistochemistry staining of the CNS section by anti-Iba1 mAb showed increased Iba1 expression upon SALM5 mAb treatment (FIGS. 3B1 and 3B2), indicating that treatment by SALM5 mAb leads to increased activation of microglia/macrophages in the CNS. Consistent to this finding, microglial cells from CNS expressed higher levels of MHC class II and CD80 in SALM5 mAb-treated mice than the control mice (FIG. 3C). The ability of freshly isolated CNS microglia/macrophages to produce proinflammatory cytokines upon SALM5 mAb treatment ex vivo was examined. Microglia/macrophages were purified from the mice treated with SALM5 or control mAb, cultured for 12 hrs without any stimulation, and the culture supernatant were measured by sandwich ELISA. The cultures from SALM5 mAb-treated mice produced significantly higher levels of pro-inflammatory cytokines including IL-6, TNF-α and IL-10 than those of control mAb (FIG. 3D). Collectively, these results support an important role of SALM5 mAb in the activation of microglia/macrophages in the CNS and implicate a suppressive role of SALM5-expressing neuronal cells in microglia/macrophage-mediated neuroinflammation.

Next to be determined was whether SALM5 mAb could promote neuroinflammation without the involvement of T cells. Systemic administration of lipopolysaccharide (LPS) is shown to induce CNS inflammation by activating microglial cells (Qin et al., 2007, Glia 55: 453-462). SALM5 mAb treatment significantly increased the activation of microglial cells, as revealed by intensified Iba1 staining in the spinal cord (FIGS. 3E1 and 3E2). Therefore, SALM5 mAb-induced activation of microglia/macrophages in the CNS may not require T cells.

To test whether SALM5 directly suppresses macrophage activation, macrophages isolated from peritoneal cavity were pre-incubated with SALM5-transfected or mock transfected HEK293T cells for 12 hrs before activation by LPS. Pro-inflammatory cytokine IL-6 and TNF-alpha in culture supernatant were measured by specific sandwich ELISA. As shown in FIG. 3F, production of both IL-6 and TNF-alpha from macrophages was inhibited significantly by SALM5+ HEK293T cells. Therefore, SALM5 directly suppresses macrophage activation, which is believed to be accomplished by having SALM5 engage with a putative receptor on a macrophage.

### HVEM is the Counter-Receptor for SALM5

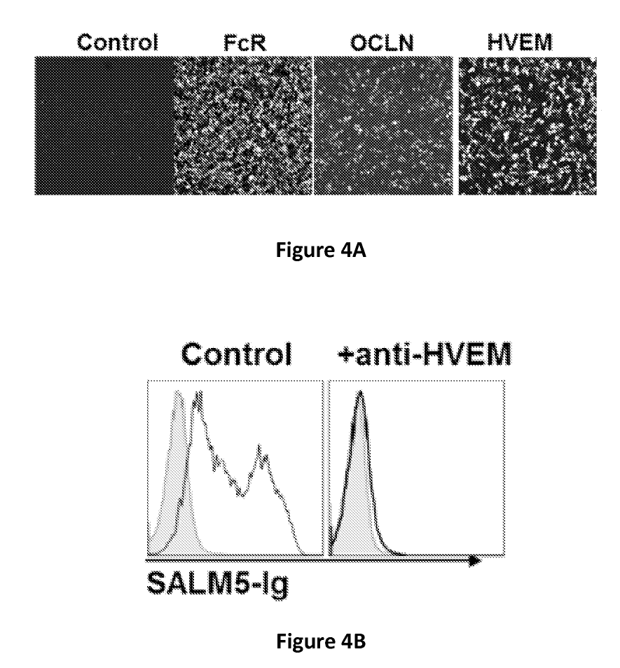

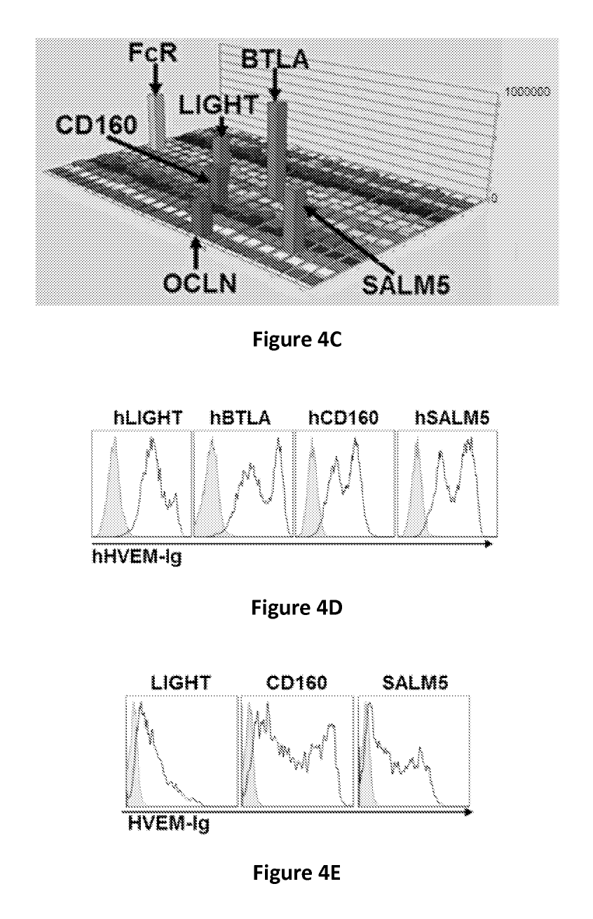

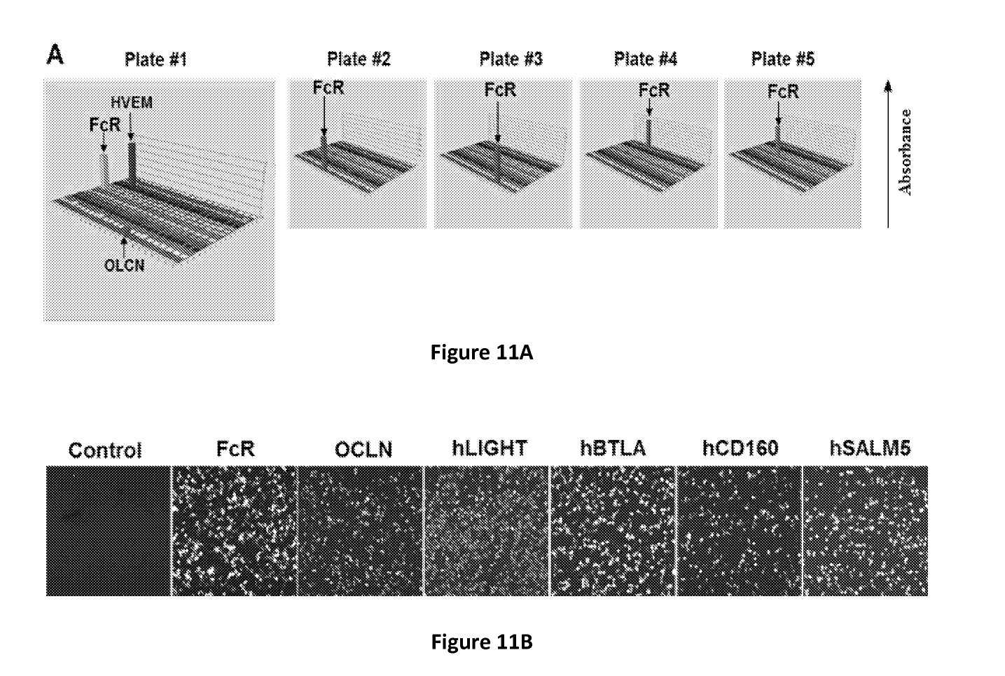

Given the potential importance of SALM5 in the control of CNS inflammation, SALM5 counter-receptor was identified by screening a receptor-ligand proteome in a high throughput format (Yao et al., 2011, Immunity 34: 729-740). Briefly, over 2,300 full length human transmembrane genes were transfected individually to be expressed on HEK293T cells. These genes include molecules from the immunoglobulin superfamily (IgSF), tumor necrosis factor superfamily (TNFRSF), C-type lectin superfamily, G protein coupled receptor (GPCR) superfamily, as well as the majority of integrins (pairs) and scavenger receptors. SALM5-Ig recombinant fusion protein was purified and employed as bait. Fluorescence-labeled anti- Fc was applied to detect the binding of SALM5-Ig to the transfected HEK293T cells and screened by 8200 Cellular Detection System (CDS) (Applied Biosystem). In addition to the wells containing FcR and OCLN as internal positive controls, one positive hit for SALM5 binding was found, which was identified as HVEM (FIGS. 4A and 11A). This interaction was further evaluated using both human and mouse SALM5 and HVEM by flow cytometry. As shown in FIG. 4B, mouse SALM5-Ig bound to mouse HVEM transfectant, but not mock transfectant. Furthermore, inclusion of a HVEM mAb completely abolished this interaction. To further verify the specificity of this interaction, we also used human HVEM-Ig fusion protein to screen the library. The well containing human SALM5 gene revealed a strong positive signal (FIGS. 4C and 11B). As expected, HVEM-Ig also bound to HEK293T cells transfected with SALM5, BTLA and CD160, all known counter-receptors for HVEM (Cai et al., 2008, Nat. Immunol. 9: 176-185; Harrop et al., 1998a, J. Biol. Chem. 273: 27548-27556; Sedy et al., 2005, Nat. Immunol. 6: 90-98). Interestingly, HVEM-Ig did not generate any positive signal in the wells containing the other four SALM family members; even though the members of SALM family share about 50% homology in protein sequences. This data thus support the specificity of the SALM5-HVEM interaction. Further validation by flow cytometry demonstrates that, in addition to SALM5, BTLA and CD160, human HVEM-Ig interacted strongly to human SALM5 transfectant, but not to mock transfectant (FIG. 4D). This binding was well conserved in different species, as mouse HVEM-Ig also strongly bound mouse SALM5 transfectant (FIG. 4E). The interaction between CD160 and HVEM was verified in mouse as reported previously (Cai et al., 2008, Nat. Immunol. 9: 176-185). All together, these results identified HVEM as a new counter-receptor for SALM5.

### Identification of Binding Domains of SALM5-HVEM Interaction

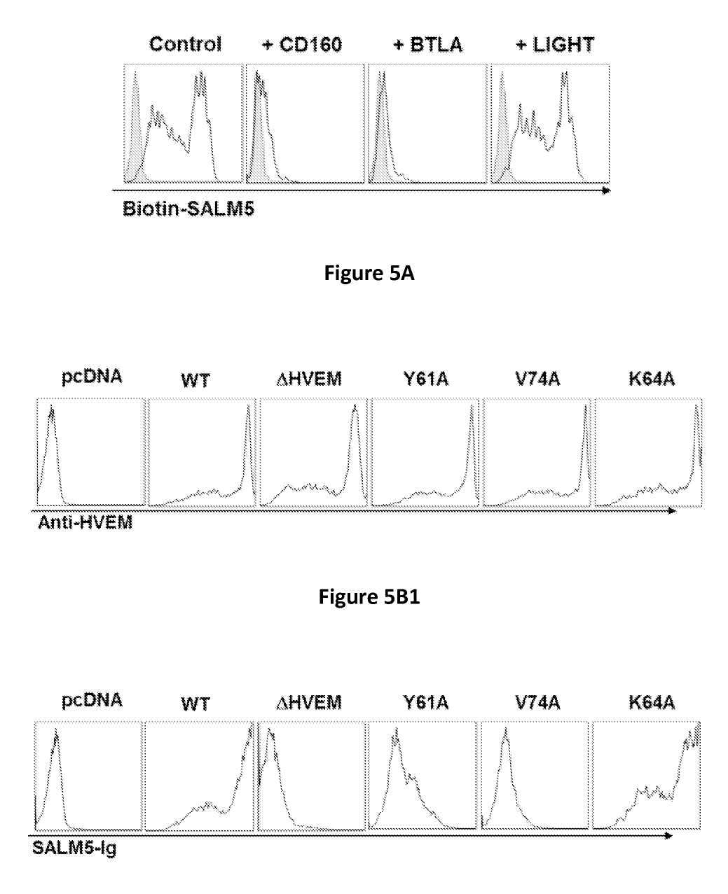

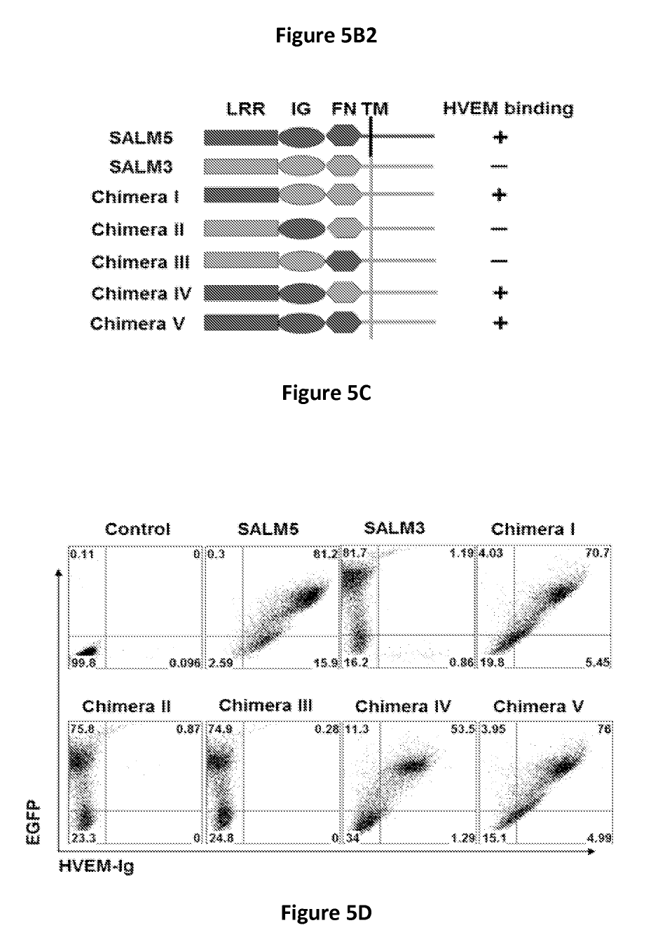

The HVEM molecule utilizes four cysteine-rich domains (CRDs) to interact with its counter-receptors (Murphy et al., 2006, Nat. Rev. Immunol. 6: 671-681; Ware, 2009, Adv. Exp. Med. Biol. 647: 146-155). BTLA and CD160, both belonging to the immunoglobulin (Ig) superfamily, are shown to bind HVEM CRD1 region, whereas SALM5, a TNF superfamily member, binds the CRD2 and CRD3 regions without interfering with BTLA or CD160 binding (Cai et al., 2009, Immunol. Rev. 229: 244-258). To dissect the interactions of SALM5 with other HVEM counter-receptors, HVEM transfectant was first incubated with BTLA, CD160 or SALM5 fusion protein and subsequently stained cells with biotin-labeled SALM5 protein. The addition of BTLA or CD160 protein completely blocked the binding of SALM5 to HVEM transfectant, while pre-inclusion of SALM5 fusion protein had minimal effect on the SALM5-Ig binding (FIG. 5A). These data indicate that SALM5 binds HVEM via the CRD1 domain.

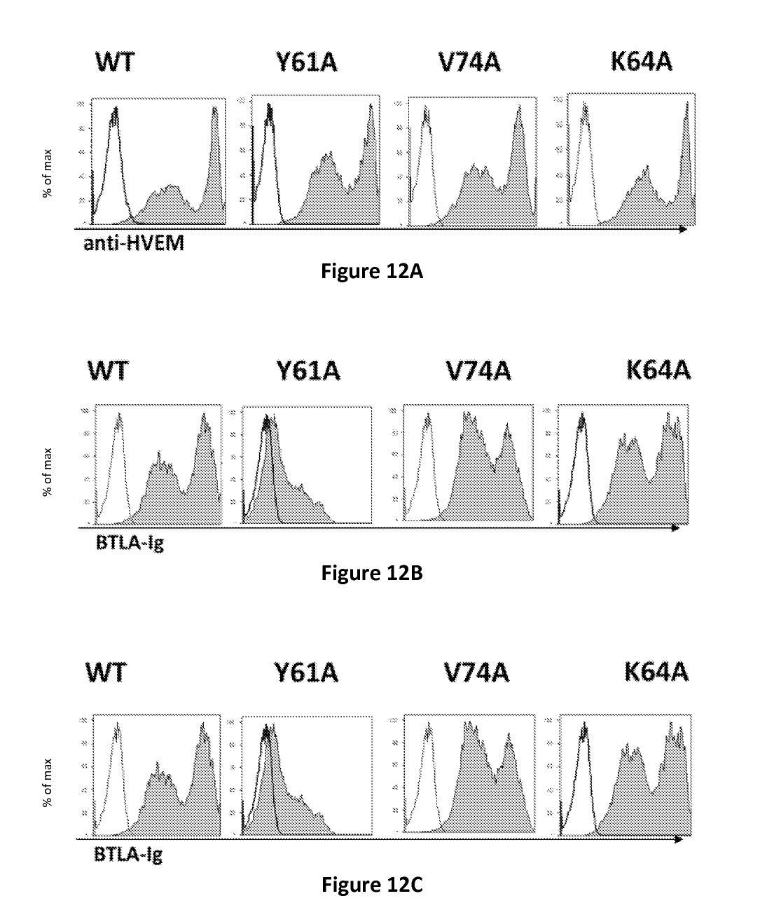

To further confirm this interaction, a HVEM deletion mutant lacking the CRD1 domain (AHVEM), and a series of HVEM point mutants within the CRD1 region which have proved to be important for the BTLA-HVEM interaction by site-directed mutagenesis was constructed (Cheung et al., 2005, Proc. Natl. Acad. Sci. USA 102: 13218-13223; Compaan et al., 2005, J. Biol. Chem. 280: 39553-39561). HEK293T cells were transfected with plasmids harboring wild-type (WT) HVEM, CRD1Δ HVEM and CRD1 mutants. Polyclonal HVEM antibodies staining confirmed similar levels of HVEM surface expression (FIG. 5B1). Deletion of CDR1 of HVEM completely eliminated the SALM5 binding, demonstrating that the CDR1 domain of HVEM is essential for the SALM5 interaction. In addition, Y61A and V74A mutants largely lost the binding to SALM5. K64A mutation, however, had only a small effect on SALM5/HVEM interaction (FIG. 5B2). Meanwhile, the same K64A mutation significantly affected the CD160-HVEM interaction with a minimal effect on the BTLA-HVEM interaction (FIGS. 12A-12C). Taken together, SALM5 interacts with the CRD1 domain on HVEM with overlapping binding site for both BTLA and CD160.

To identify the binding domain on SALM5, a series of SALM5 mutants were generated for analysis. Since HVEM does not bind to other SALM family members, each SALM5 domain was substituted with a corresponding portion from SALM3, and these mutants fused with a C-terminal EGFP tag. The intensity of GFP fluorescence reflects the expression level of the chimeras upon transfection into HEK293T cells. As expected, HVEM interacted with HEK293T cells transfected with SALM5 but not SALM3. Interestingly, the LRR domain, but not Ig, FN or transmembrane/intracellular domain from SALM5 is sufficient to endow the binding capacity to HVEM (FIGS. 5C and 5D), demonstrating that SALM5 binds HVEM through its LRR domain.

### SALM5 mAb Aggravates EAE by Blocking SALM5-HVEM Interaction

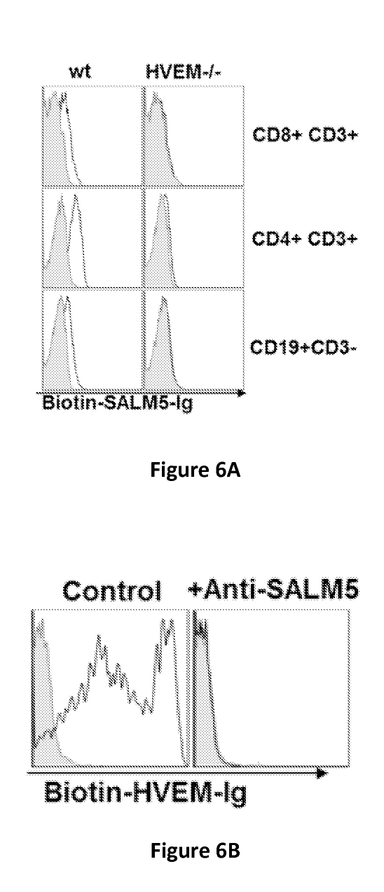

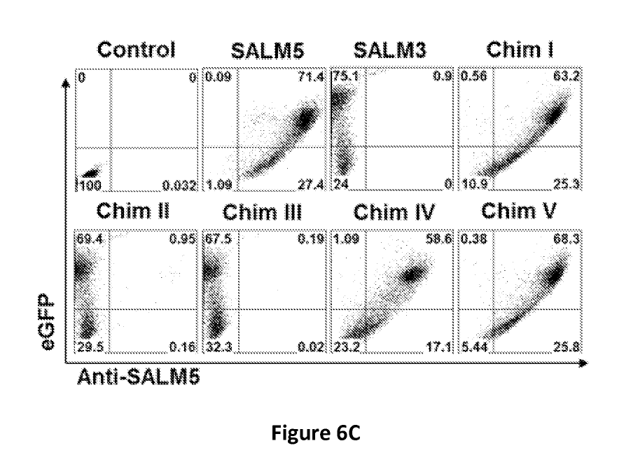

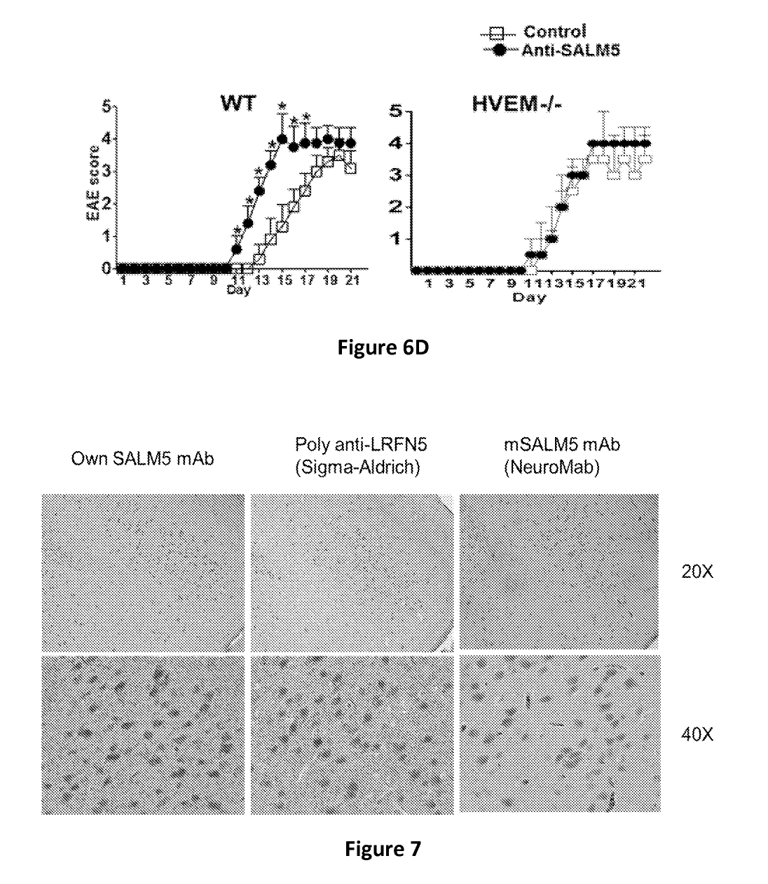

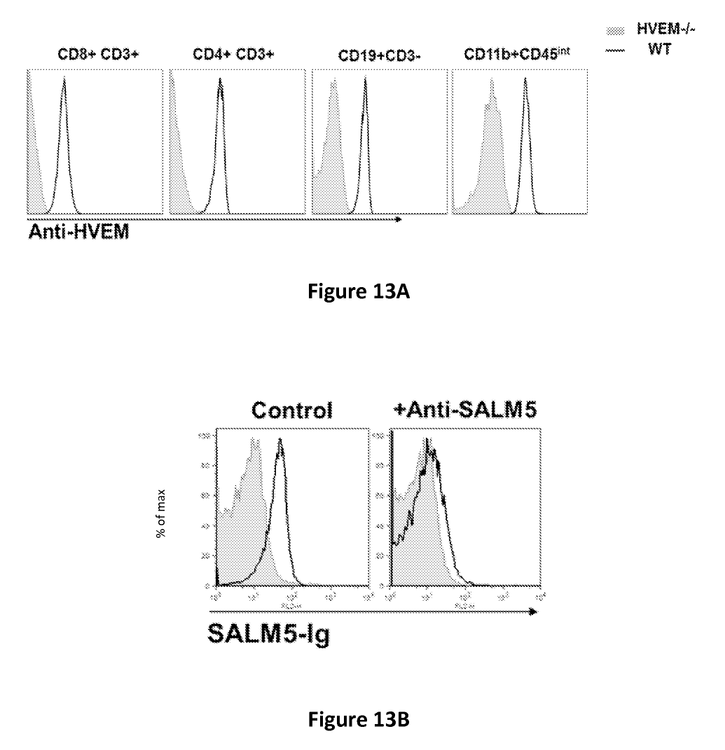

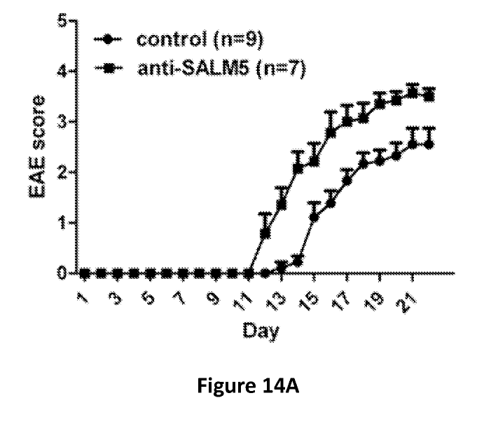

Having established that HVEM is a counter-receptor for SALM5, the next experiments were designed to determine whether the effect of SALM5 mAb is mediated by blocking SALM5/HVEM interaction. SALM5-Ig bound freshly isolated splenic CD4+, CD8+ T cells and CD19+ B cells from wild type mice, consistent with the expression pattern of HVEM (Harrop et al., 1998b, J. Immunol. 161: 1786-1794) (FIGS. 13A and 13B). This binding was completely abrogated when splenocytes from HVEM−/− mice were tested, suggesting that HVEM is the major counter-receptor for SALM5 on T and B lymphocytes (FIG. 6A). Importantly, inclusion of the SALM5 mAb 7A10 completely blocked the binding of HVEM-Ig to SALM5+ HEK293T cells (FIG. 6B), indicating that the SALM5 mAb is a blocking antibody for SALM5/HVEM interaction. In addition, this antibody blocked the binding of SALM5-Ig to freshly isolated microglia (FIG. 13B). To further validate this finding, the binding site of SALM5 mAb was determined using flow cytometry analysis of HEK293T cells transfected with each chimeric SALM5 molecule as described in FIG. 5C. As shown in FIG. 6C, the SALM5 mAb bound to the chimera I, IV and V which possess the intact LRR domain of SALM5. In contrast, the chimeric SALM5 (II and III) in which the SALM5-derived LRR domain was replaced by the SALM3-derived LRR domain (FIG. 5C), completely lost the binding by the SALM5 mAb. These results indicate that the SALM mAb binds to the LRR domain of SALM5, which also is the interacting site for HVEM. The data presented herein thus demonstrate that SALM mAb 7A10 is a specific blocking antibody for SALM5-HVEM interaction.

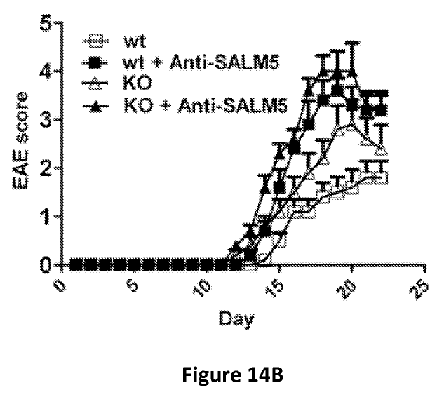

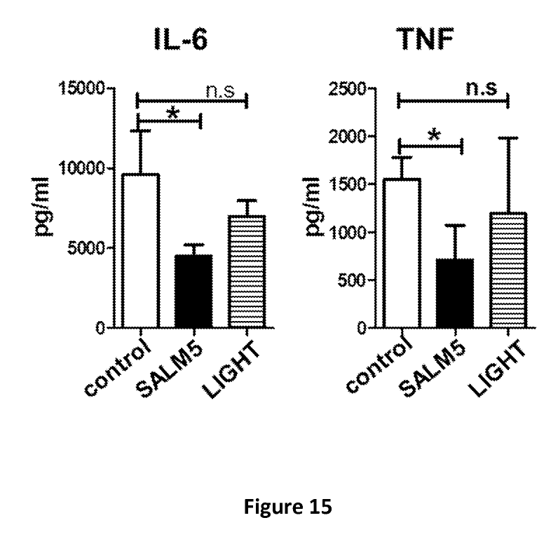

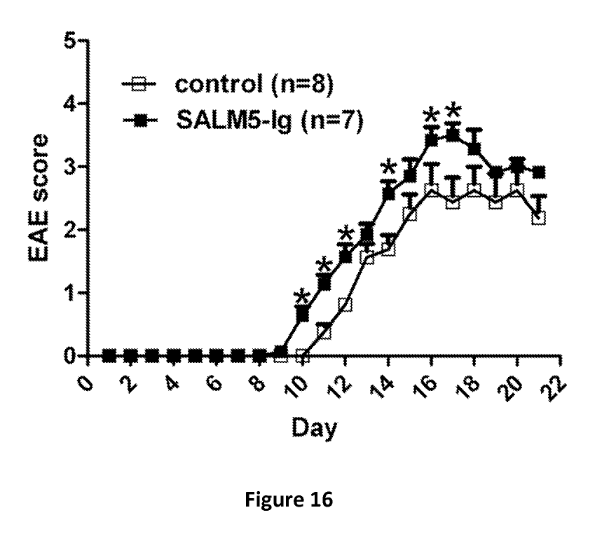

Although not wishing to be bound by any particular theory, it is believed that if the aggravating effect of the SALM5 mAb in EAE is due to blocking the endogenous SALM5-HVEM interaction, it would be expected that the effect of this mAb would rely on the expression of HVEM. To test this hypothesis, EAE was first induced in HVEM−/− mice, and subsequently the mice were treated with the SALM5 mAb. As shown in FIG. 6D, the SALM5 mAb was ineffective in HVEM−/− mice, while the identical treatment of wild type mice with this mAb aggravated EAE disease in terms of both disease onset and peak severity. It was concluded that the effect of SALM5 mAb on EAE is dependent on endogenous HVEM, and the role of this mAb in vivo is to block the SALM5-HVEM interaction. HVEM on T cells seems to be dispensable for the effect on SALM5 mAb because SALM5 mAb could still exacerbate EAE disease in a model where purified HVEM-deficient 2D2 transgenic T cells were transferred to induce EAE diseases (FIG. 14B). Although not wishing to be bound by any particular theory, these data suggest an obstruction of the SALM5-HVEM interaction accelerates the progression and aggravates the severity of EAE, demonstrating an essential role of SALM5-HVEM interaction in controlling neuroinflammation in this model.

### Role of SALM Family Molecules in CNS Immune Privilege

Reported herein is the identification and characterization of an organ-specific pathway, which modulates inflammatory immune responses in the CNS. In this pathway, SALM5, a molecule found mainly on neuronal cells, interacts with HVEM on myeloid cells to suppress inflammation in the CNS during ongoing neuroinflammation. These findings uncover a molecular mechanism in the negative regulation and control of neuroinflammation and provide an interpretation for a long-standing puzzle for the immune privilege of CNS. Importantly, these findings provide potential therapeutic targets for the treatment of inflammatory neurological diseases.

Expression profiling in either mRNA or protein by various laboratories demonstrates that SALM5 molecule is highly organ-specific. While both mRNA and protein could be detected in the brain and embryo during late development, peripheral organs including heart, spleen, lung, liver, skin, skeletal muscle and kidney are all negative for SALM5 expression (Ko et al., 2006, Neuron 50: 233-245; Mah et al., 2010, J. Neurosci 30: 5559-5568; Morimura et al., 2006, Gene 380: 72-83). Another potential expression site for this protein is testis where a weak signal could be detected in Western blot analysis in a recent study (Mah et al., 2010, J. Neurosci 30: 5559-5568). These findings have been validated using a highly sensitive RT-PCR method. Specific SALM5 mRNA were not detected in testis and tissues other than brain and embryo (FIG. 1A), which is consistent with the previous results using Northern blotting (Morimura et al., 2006, Gene 380: 72-83). In addition, immunostaining further confirmed that SALM5 protein is constitutively expressed in the CNS but not spleen. The expression of SALM5 protein was predominantly found within the synaptic fraction of neurons (Mah et al., 2010, J. Neurosci 30: 5559-5568). With this highly restricted expression pattern, and without wishing to be bound by any particular theory, it is believed that SALM5 may function during development as well as in the adult brain. In fact, SALM5 is shown to be a critical adhesion molecule in the regulation of neurite growth and synapse formation (Mah et al., 2010, J. Neurosci 30: 5559-5568; Wang et al., 2008, Mol. Cell Neurosci. 39: 83-94). The receptor library used in the present study contains about 2,300 membrane proteins, which is about half of transmembrane genes predicted from the human genome database (Yao et al., 2011, Immunity 34: 729-740). Therefore, though screening revealed HVEM as the sole binding partner for SALM5, it is still possible that unknown counter-receptors for SALM5 exist. These findings thus reveal an unexpected role of SALM5 in taming neuroinflammation. The results presented herein demonstrate that in a mouse EAE model, blockade of the SALM5-HVEM pathway aggravated disease. Consistent to this observation, enhanced immune cells infiltration and proinflammatory cytokines were found in the CNS upon SALM5 blockade, which seems to be largely contributed by excessive activation of the macrophages.

A stunning finding in this study is that systemic administration of SALM5 mAb does not affect T cell activation outside the CNS (FIGS. 2A-2G). In a mouse EAE model, immunization of B6 mice with MOG (35-55) peptide, which encodes a CD4+ T cell epitope on myelin oligodendrocyte glycoprotein, generates a strong immune response mediated by CD4+ T cells and leads to paralysis of mice due to neuroinflammation and demyelination. SALM5 mAb could aggravate EAE without affecting T cell priming. This finding might be interpreted in part by the lack of expression of SALM5 in peripheral lymphoid organs. However, other factors might be also involved because in vitro SALM5 engagement of naïve or activated T cells, which do constitutively express HVEM, did not suppress T cell activation (FIG. 10). In this study, macrophages and microglial cells, but not T cells, appear to be the target cells for SALM5. Constitutive expression of SALM5 in neuronal cells should allow this interaction to take place as microglial cells constitutively express HVEM (FIG. 13). A recent study nicely revealed the contact between neuron cells and microglia in the CNS via quantitative electron microscopy and two-photon in vivo imaging (Tremblay et al., 2010, PLoS Biol. 8, e1000527). In the context of constitutive expression of SALM5 in neuronal cells and HVEM on microglial cells, the results presented herein support that SALM5-HVEM interaction constitutes a default pathway to provide a suppressive mechanism to maintain immune privilege of CNS.

Studies by competitive binding and mutagenesis revealed several important features of the interaction between SALM5 and HVEM. First, the SALM5-HVEM interaction is shown to be highly specific as well as species-conserved. The SALM family has five molecules that have been characterized. The sibling SALM family members, though sharing ˜50% protein sequence identity with SALM5, do not bind HVEM, indicating a conserved interaction. The second important feature is that SALM5 interacts with a “suppressive domain” on HVEM. HVEM has been shown to interact with multiple molecules to execute various immune modulatory functions, from lymphocyte costimulation to T cell suppression. Functional diversity of this molecule relies on interactions between its different domains and distinct ligands. The CRD1 domain has been shown to interact with BTLA and CD160 to inhibit T cell response (Cai et al., 2008; Sedy et al., 2005), though the directionality of signaling underlying these interactions has yet to be elucidated. On the other hand, the CDR2/3 domains of HVEM are shown to bind SALM5 and LTα and transmit a costimulatory signal to T cells (Tamada et al., 2000, Nat. Med. 6: 283-289; Watts et al., 2005, Proc. Natl. Acad. Sci. USA 102: 13365-13366). These studies unambiguously demonstrate that SALM5 interacts with the CRD1 domain of HVEM and competes with the binding of BTLA and CD160 without interfering with SALM5 interaction. In the context of inhibitory functions by SALM5 as well as by BTLA and CD160, the CRD1 domain of HVEM appears to be exclusively suppressive. The third feature is the unique HVEM-binding domain within SALM5 molecule. Despite competing with two Ig superfamily molecules, BTLA and CD160 for HVEM binding, SALM5 interacts with HVEM through its LRR domain but not its IgC-like domain. This is the first molecular evidence that a TNFR member binds a LRR domain. It appears that HVEM has evolved with high flexibility to interact with various molecular motifs.

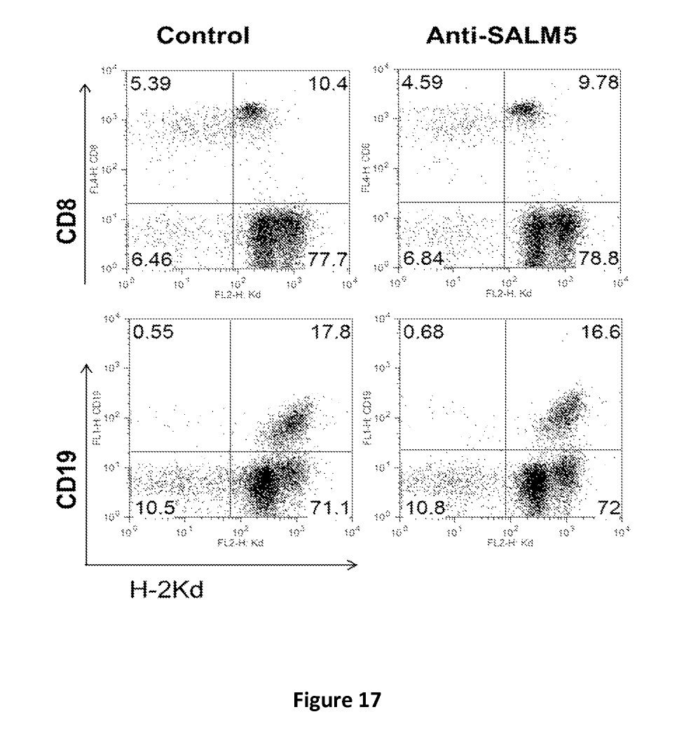

While the identification of SALM5 as a new counter-receptor for HVEM helps to better understand the complex functions of HVEM, these findings also provide a unique opportunity to modulate inflammation and immune responses in the CNS. Due to interactions of HVEM with multiple immune modulatory molecules, it is difficult to target HVEM for therapeutic manipulation and such manipulation often leads to confusing and sometimes contradictory findings. For example, HVEM−/− mice were hypersensitive to Concanavalin A-induced hepatitis, and more susceptible to the induction of EAE accompanied with enhanced T cell response (Wang et al., 2005, J. Clin. Invst. 115: 711-717). However, T cells from HVEM−/− mice were mild inducers in allogeneic GVHD and inflammatory bowel disease, which are often associated with suppressed T cell responses (Steinberg et al., 2008, J. Exp. Med. 205: 1463-1476; Xu et al., 2007, Blood 109: 4097-4104). In this study, blockade of SALM5 in EAE had a similar phenotype as observed with the loss of HVEM. In addition, the same antibody used to block SALM5 did not affect allogeneic GVHD while other known HVEM ligands did (FIG. 17) (Sakoda et al., 2011, Blood 117: 2506-2514). In addition, ablation of HVEM eliminates the effect of SALM5 mAb in EAE (FIG. 6D). Although not wishing to be bound by any particular theory, these seemingly contradictory data could be interpreted with the availability of HVEM counter-receptors rather than HVEM itself. On the other hand, disruption of SALM5 and HVEM interaction by specific mAb may be utilized to enhance inflammatory immune responses against neoplasm and viral infection in the CNS or other immune privileged tissue. Manipulation of HVEM counter-receptors rather than HVEM thus may represent a highly selective approach to modulate inflammation and immune response. The results presented herein open a new avenue for the prevention and treatment of inflammatory and immune diseases and malignancies in the CNS.

The disclosures of each and every patent, patent application, and publication cited herein are hereby incorporated herein by reference in their entirety.

While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. A composition comprising a modulator of inflammation in an immune-privileged organ.

2. The composition of claim 1, wherein the composition binds to herpes virus entry mediator (HVEM).

3. The composition of claim 1, wherein the composition binds to a synaptic adhesion-like molecule (SALM).

4. The composition of claim 1, wherein the composition prevents the binding of SALM with HVEM.

5. The composition of claim 3, wherein the SALM is SALM5.

6. The composition of claim 1, wherein the composition is at least one selected from the group consisting of a small molecule drug, a polypeptide, a peptide, and an antibody.

7. The composition of claim 6, wherein the polypeptide is at least one selected from the group consisting of a synaptic adhesion-like molecule (SALM) and a fragment of a SALM.

8. The composition of claim 6, wherein the polypeptide is at least one selected from the group consisting of SALM5 and a fragment of a SALM5.

9. The composition of claim 6, wherein the antibody is an anti-HVEM antibody.

10. The composition of claim 1, wherein the composition binds to the CRD1 domain of HVEM.

11. The composition of claim 6, wherein the antibody is an anti-SALM5 antibody.

12. A method of diminishing inflammation in an immune-privileged organ of a subject in need thereof, comprising: contacting a cell of the subject with a composition comprising an agonist, wherein the agonist agonizes at least one selected from the group consisting of a synaptic adhesion-like molecule (SALM), a herpes virus entry mediator (HVEM) and the interaction between a SALM and a HVEM.

13. The method of claim 12, wherein the composition is at least one selected from the group consisting of: a small molecule drug, a nucleic acid molecule a polypeptide, a peptide, and an antibody.

14. The method of claim 13, wherein the polypeptide is at least one selected from the group consisting of: SALM and a fragment of SALM.

15. The method of claim 13, wherein the polypeptide is at least one selected from the group consisting of: SALM5 and a fragment of SALM5.

16. The method of claim 13, wherein the antibody is an anti-HVEM antibody and mimics the activity of SALM5 on HVEM when the antibody is bound to HVEM.

17. The method of claim 12, wherein the composition binds to the CRD1 domain of HVEM and mimics the activity of SALM5 on HVEM.

18. The method of claim 12, wherein the immune-privileged organ is at least one selected from the group consisting of: CNS, testes, placenta and eye.

19. The method of claim 12, wherein the cell is a lymphoid cell or a myeloid cell.

20. A method of increasing inflammation in an immune-privileged organ of a subject in need thereof, comprising: contacting a cell of the subject with a composition comprising an antagonist, wherein the antagonist prevents the binding of SALM with HVEM.

21. The method of claim 20, wherein the SALM is SALM5.

22. The method of claim 20, wherein the composition is at least one selected from the group consisting of: a small molecule drug, a polypeptide, a peptide, and an antibody.

23. The method of claim 22, wherein the antibody is an anti-SALM5 antibody.

24. The method of claim 23, wherein the antibody binds to the CRD1 domain of HVEM.

25. The method of claim 22, wherein the peptide comprises the LRR domain of SALM5.

26. The method of claim 22, wherein the polypeptide comprises the LRR domain of SALM5.

27. The method of claim 20, wherein the immune-privileged organ is at least one selected from the group consisting of: CNS, testes, placenta and eye.

28. The method of claim 20, wherein the cell is a cell of the immune-privileged organ.

29. The method of claim 20, wherein the subject has cancer.

30. The method of claim 29, wherein increasing inflammation in an immune-privileged organ of the subject is used to treat cancer in the subject.

31. The method of claim 20, wherein the subject has an infection.

32. The method of claim 31, wherein increasing inflammation in an immune-privileged organ of the subject is used to treat infection in the subject.
